(19) United States
(12) Patent Application Publication
(10) Pub. No.: US 2011/0301431 A1
(43) Pub. Date: Dec. 8, 2011

(54) Methods of classifying cognitive states and traits and applications thereof

(75) Inventors: Michael D. Greicius, Palo Alto, CA (US); Vinod Menon, Los Altos, CA (US)

(73) Assignee: The Board of Trustees of the Leland Stanford Junior University, Palo Alto, CA (US)

(21) Appl. No.: 13/153,465

(22) Filed: Jun. 5, 2011

Related U.S. Application Data
(60) Provisional application No. 61/351,886, filed on Jun. 5, 2010.

(51) Int. Cl. A61B 5/00

(52) U.S. Cl. 600/300

(57) ABSTRACT

The present invention provides for improved brain imaging and decoding methods that test subjects under authentic, natural conditions that allow for regular patterns of free-flowing thought and perception, as they occur in everyday life, while taking into account brain activities that were measured over spatially diverse regions of the whole-brain (whole-brain connectivity signatures). From such whole-brain connectivity signatures, specific cognitive traits and states are decoded and classified in a whole-brain connectivity analysis which takes into account the full pattern of brain activity. Such methods find applications in clinical diagnosis and monitoring of neuropsychiatric diseases and in nonclinical areas such as neuromarketing and neuroeconomics.

### RELATED APPLICATION

This application claims priority and other benefits from U.S. Provisional Patent Applications Ser. No. 61/351,886, filed Jun. 5, 2010, entitled “Methods of classifying cognitive states or traits and applications thereof”. Its entire content is incorporated herein by reference.

### STATEMENT OF GOVERNMENT SUPPORT

This invention was made with government support under NSO48302 awarded by the National Institutes of Health. The government has certain rights in the invention.

### TECHNICAL FIELD OF THE INVENTION

The present invention relates to the field of brain imaging and, in particular, to the assessment and classification of cognitive traits and states from brain imaging data for use in neuropsychiatric disease diagnosis, monitoring of disease progression as well as disease treatment success. The present invention relates, furthermore, to the classification of cognitive states from brain imaging data for application in neuromarketing and neuroeconomics.

### BACKGROUND

Decoding specific cognitive and perceptual states from brain activity where one could come close to reading another individual's mind constitutes a major and still unattained goal of neuroscience. Similarly, clinical neuroscience would benefit tremendously from the ability to read out a subject's diagnosis based on his brain activity. Two of the most challenging topics facing the successful application of neuroimaging methods in this context are the conditions under which brain activity measurements are typically taken from a subject and the selection of brain regions from which those brain activity measurements are taken.

Conventional neuroimaging approaches compartmentalize the brain into tens of thousands of arbitrarily divided cubes known as voxels and compare brain activity in a voxel during a given state of interest to brain activity during a second “control” state. This comparison is done for each voxel resulting in tens of thousands of comparisons raising the likelihood of false positive findings due to multiple comparisons. Such approaches require artificial experimental conditions such as frequent, precisely timed switching between the cognitive state of interest and the control state.

It would be highly desirable to have neuroimaging and decoding methods available that benefited from a parcellation of the brain into functionally defined regions-of-interest (rather than arbitrary cubes), that could detect patterns of brain connectivity across multiple brain regions at once (rather than activity on a voxel by voxel basis), thereby allowing for the characterization of more naturalistic, free-flowing states of thought and perception. Such an approach would allow for the analysis of more “real-world” thought processing and be applicable in various populations of subjects suffering from neuropsychiatric diseases and disorders who are difficult to assess with traditional functional brain imaging.

### SUMMARY

The present invention provides for improved brain imaging and decoding methods which test subjects under authentic, natural conditions that allow for regular patterns of free-flowing thought and perception, as they occur in everyday life, while measuring brain connectivity across a set of functionally-defined brain regions covering the whole-brain (whole-brain connectivity signatures). From such whole-brain connectivity analyses, specific cognitive traits and states are decoded and classified based on their whole-brain connectivity signature.

In one aspect of the present invention, a subject's brain connectivity is measured by brain imaging across a plurality of functionally-defined regions of interest in a continuous, free-streaming manner with uninterrupted brain imaging scan periods ranging from several seconds to several minutes in length. Free-streaming, subject-driven mental states account for most of human conscious processing (James, 1918).

In another aspect of the invention, specific cognitive and perceptual states are decoded and classified from a subject's whole-brain connectivity signature derived from a whole-brain connectivity analysis which takes into account the full pattern of brain connectivity.

In one aspect of the present invention, the identification of specific whole-brain connectivity signatures is used to diagnose a neuropsychiatric disease or disorder. In one embodiment of the present invention, the classification of specific cognitive traits is used to diagnose a neurodegenerative disease such as Alzheimer's disease, Parkinson's disease, Lewy body dementia, Huntington's disease, a tauopathy, a serpinopathy, a prion disease, frontotemporal or vascular dementia. In another embodiment of the present invention, the classification of specific cognitive traits is used to diagnose chronic pain. In yet another embodiment of the present invention, the classification of specific cognitive traits is used to diagnose depression. In a further embodiment of the present invention, the classification of specific cognitive traits is used to diagnose anxiety.

In another aspect of the present invention, the classification of specific cognitive traits is used to monitor progression of a neuropsychiatric disease or disorder. In one embodiment of the present invention, the classification of specific cognitive traits is used to monitor the progression of a neurodegenerative disease such as Alzheimer's disease, Parkinson's disease, Lewy body dementia, Huntington's disease, a tauopathy, a serpinopathy, a prion disease, frontotemporal or vascular dementia. In another embodiment of the present invention, the classification of specific cognitive traits is used to monitor the progression of chronic pain. In yet another embodiment of the present invention, the classification of specific cognitive traits is used to monitor the progression of depression. In a further embodiment of the present invention, the classification of specific cognitive traits is used to monitor the progression of anxiety.

In another aspect of the present invention, the classification of specific cognitive traits is used to monitor treatment success of a neuropsychiatric disease or disorder. In one embodiment of the present invention, the classification of specific cognitive traits is used to monitor treatment success of a neurodegenerative disease such as Alzheimer's disease, Parkinson's disease, Lewy body dementia, Huntington's disease, a tauopathy, a serpinopathy, a prion disease, frontotemporal or vascular dementia. In another embodiment of the present invention, the classification of specific cognitive traits is used to monitor treatment success of chronic pain. In yet another embodiment of the present invention, the classification of specific cognitive traits is used to monitor treatment success of depression. In a further embodiment of the present invention, the classification of specific cognitive traits is used to monitor treatment success of anxiety.

In a further aspect of the present invention, the classification of specific cognitive states finds applications in neuromarketing and neuroeconomics to predict consumer behavior and financial decision making.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates functional parcellation of the brain into 90 regions-of-interest (ROIs) that cover the majority of cortical and subcortical gray matter. Group independent component analysis (ICA) was applied to the resting-state data of 15 subjects yielding 14 intrinsic connectivity networks (ICNs) of which 5 are shown in panel A; all fourteen ICNs are shown in FIG. 2. Each ICN is thresholded to generate between 2 and 12 ROIs per network. When all 90 ROIs across the 14 ICNs are overlaid on a single brain image (panel B), the majority of cortical and subcortical gray matter is covered. X, y, and z indicate the different spatial dimensions of imaging.

FIG. 2 illustrates all fourteen intrinsic connectivity networks (ICNs) that were identified in resting-state data by group ICA. This figure shows the ROIs contained within each ICN. The ICNs are presented in the same order as they appear on the axes of FIG. 3A. (A) Auditory, (B) Basal Ganglia, (C) Posterior Cingulate Cortex (PCC)/Medial Prefrontal Cortex (MPFC), (D) Secondary Visual Cortex (V2), (E) Language, (F) Left Dorsolateral Prefrontal Cortex (DLPFC)/Left Parietal Lobe, (G) Sensorimotor, (H) Posterior Insula, (I) Precuneus, (J) Primary Visual Cortex (V1), (K) Right Dorsolateral Prefrontal Cortex (DLPFC)/Right Parietal Lobe, (L) Insula/Dorsal Anterior Cingulate Cortex (dACC), (M) Retrosplenial Cortex (RSC)/Medial Temporal Lobe (MTL), (N) Intraparietal Sulcus (IPS)/Frontal Eye Field (FEF).

FIG. 3 illustrates that subject-driven episodic memory recall drives changes in whole-brain functional connectivity. A single subject's connectivity matrix is shown for the rest scan in panel A. Cells colored in red-yellow indicate a positive pairwise correlation between two ROIs; blue-green cells indicate negative pairwise correlations. Coarse anatomic labels for each ICN are indicated along the x- and y-axes; more detailed anatomic information is available in Table 1. Each network is bracketed by black bars and divided into 2-12 ROIs. The strong within-ICN correlations are evident along the diagonal. The same subject's memory state connectivity matrix is shown in panel B. Subtracting the rest state matrix from the memory state matrix provides the difference matrix shown in panel C where connectivity within the retropslenial cortex/medial temporal lobes (RSC/MTL network) is shown to increase during the memory task. A paired-sample t-test of the state matrices across all fourteen subjects (panel D) reveals changes in connectivity both between and within ICNs. These within-ICN changes (orange arrow) can also be detected by performing a paired-sample t-test on the individual subject ICA data (panel E). This analysis reveals clusters in the RSC/MTL network whose connectivity increases significantly during the memory scan compared to the rest scan.

FIG. 4 illustrates distinct across-subject patterns of whole-brain connectivity for four subject-driven cognitive states. For each of the four states, cells of interest which showed significant, state-specific positive or negative correlations were included in the group-level state matrix. These state matrices are shown in panels A-D. The orange arrow in panel B indicates strong connectivity within the RSC/MTL network in the group-level memory state matrix. In the subtraction task (panel C) connectivity within the IPS/FEF ICN is increased (blue arrow) but the classification algorithm also highlights increased connectivity between this ICN and the basal ganglia ICN (green arrow, panel D).

FIG. 5 illustrates a flow chart of the classification algorithm that was employed in embodiments of the present invention. This flow chart illustrates the sequence of analyses that was performed on the data. We calculated pairwise correlations between 90 different ROIs (panel A), normalized the correlation coefficients using Fisher's R to Z transformation (panel B), performed a one-sample t-test with the training data for each state matrix (panel C), identified cells that were significant and unique to each state matrix (panel D), masked individual scan matrices in the test dataset with each group-level state matrix (panel E), and calculated the fit score for each individual scan to each group-level state matrix (panel F).

FIG. 6 illustrates that classification accuracy remains high with scans as short as one minute. The classification algorithm was tested initially on the full 10-minute scans but then on increasingly shorter scan lengths. In each case the shorter scan lengths are taken from the beginning of the scan (i.e., 0.5 minutes refers to the first 30 seconds of the scan). Eleven different scan lengths from 30 seconds to 10 minutes were evaluated. The orange line refers to the overall accuracy in distinguishing all four cognitive states. Accuracy for individual states is shown in the other four colors. An accuracy of 25% reflects chance level classification. The overall accuracy remains at 80% with just one minute of data. With scan lengths below one minute, overall accuracy tends to decrease, though all four scans were identified with significant accuracy with only 30 seconds of data (p<0.001).

FIG. 7 illustrates significant correlations in the spatial navigation task. A one-sample t-test of the independent cohort's spatial navigation state matrices reveals significant positive and negative connectivity across the 90 ROIs (p<0.01, corrected).

FIG. 8 illustrates group-level results for classification fit scores . Each colored bar depicts the mean fit score (10 subjects in the independent dataset) for each scan type (x-axis) with each state matrix. For each scan, the fit score to the conjugate state matrix was significantly higher than the fit score to the other three state matrices (gold asterisks).

FIG. 9 illustrates classifier accuracy when forcing unique assignment of individual scans. When forcing unique assignment of individual scans (“winner-take-all” approach”), classification reached 100% accuracy for the full 10 minutes of scan data, and remained as high as 95% with only 2 minutes of scan data.

FIG. 10 illustrates classifier specificity. Each colored bar depicts the mean fit score (10 subjects in the independent dataset) for each scan type (x-axis) with each state matrix. Each state matrix was a significantly better fit to its conjugate scan than to the spatial navigation scan.

FIG. 11 illustrates that functional ROIs outperform structural ROIs. We performed classification with 112 structural ROIs from the AAL Atlas, and 90 functional ROIs identified by ICA on resting-state data from an independent sample. Classification was performed with both sets of ROIs at 11 different scan lengths. In all comparisons, classification with functional ROIs was substantially more accurate than classification with the AAL Atlas ROIs.

FIG. 12 illustrates whole-brain functional connectivity classification of subjects suffering from Alzheimer's disease. Using a similar approach to that outlined in FIG. 5, whole-brain resting-state connectivity matrices are defined for a group of subjects suffering from Alzheimer's disease and a group of healthy older control subjects using one-sample t-tests (A). These group-level connectivity matrices are thresholded (B) and cells that appear in both matrices are removed (C). A single-subject's whole-brain resting-state functional connectivity matrix is then compared to each of the group-level matrices allowing us to calculate a fit score for each subject (D). A given subject is classified as a control if their fit score to the control matrix is greater than their fit score to the Alzheimer's matrix (difference score>0). If their difference score is less than zero (better fit to the Alzheimer's matrix) then they are classified as a subject suffering from Alzheimer's disease. Using this approach 85% of subjects are correctly classified (E).

FIG. 13 illustrates that whole-brain functional connectivity analysis detects response to donepezil in a small group of subjects suffering from Alzheimer's disease. Subjects underwent resting state fMRI before and 6 weeks after treatment with donepezil (Aricept®), a centrally acting reversible acetylcholinesterase inhibitor, used for the palliative treatment of mild to moderate Alzheimer's disease. The figure shows a paired-sample t-test of the whole-brain connectivity matrix identifying regions that had significantly increased (blue cells) or decreased (red cells) connectivity following treatment with donepezil. The grey triangle ASB-029UTL Non-provisional Patent Application Stanford ref. S10-142 highlights regions in a brain network targeted by Alzheimer's disease whose connectivity increased after treatment.

FIG. 14 illustrates that whole-brain functional connectivity analysis detects response to Sinemet® in a small group of subjects suffering from Parkinson's disease. Subjects suffering from Parkinson's disease were scanned during treatment with Sinemet® and off treatment with Sinemet®, a carbidopa/levadopa combination to treat Parkinson's disease. The figure shows a paired-sample t-test of the whole-brain connectivity matrix identifying regions that had significantly increased (red cells) or decreased (blue cells) connectivity following treatment with sinemet. The green arrows highlight cells which reflect increased connectivity between the bilateral basal ganglia and the prefrontal cortex when subjects were on Sinemet®.

FIG. 15 illustrates that whole-brain functional connectivity analysis detects response to citalopram in a small group of subjects suffering from depression. The figure shows a paired-sample t-test of the whole-brain connectivity matrix identifying regions that had significantly increased (blue cells) or decreased (red cells) connectivity following treatment with citalopram (Celexa®), a selective serotonin reuptake inhibitor, used to treat depression. The grey triangle highlights regions in a medial temporal lobe memory network whose connectivity increased after treatment.

FIG. 16 illustrates that whole-brain functional connectivity analysis detects response to duloxetine in a small group of subjects suffering from chronic pain. The figure shows a paired-sample t-test of the whole-brain connectivity matrix identifying regions that had significantly increased (blue cells) or decreased (red cells) connectivity in subjects suffering from back pain who were treated with duloxetine compared to the same subjects suffering from back pain treated with placebo. Duloxetine (Cymbalta®) is a non-narcotic, non-NSAID pain relieving agent that is indicated, among other indications, for chronic musculoskeletal pain. The green arrows identify cells that reflect increased connectivity between bilateral sensory regions and the thalamus in subjects suffering from back pain, when treated with duloxetine compared to when treated with placebo.

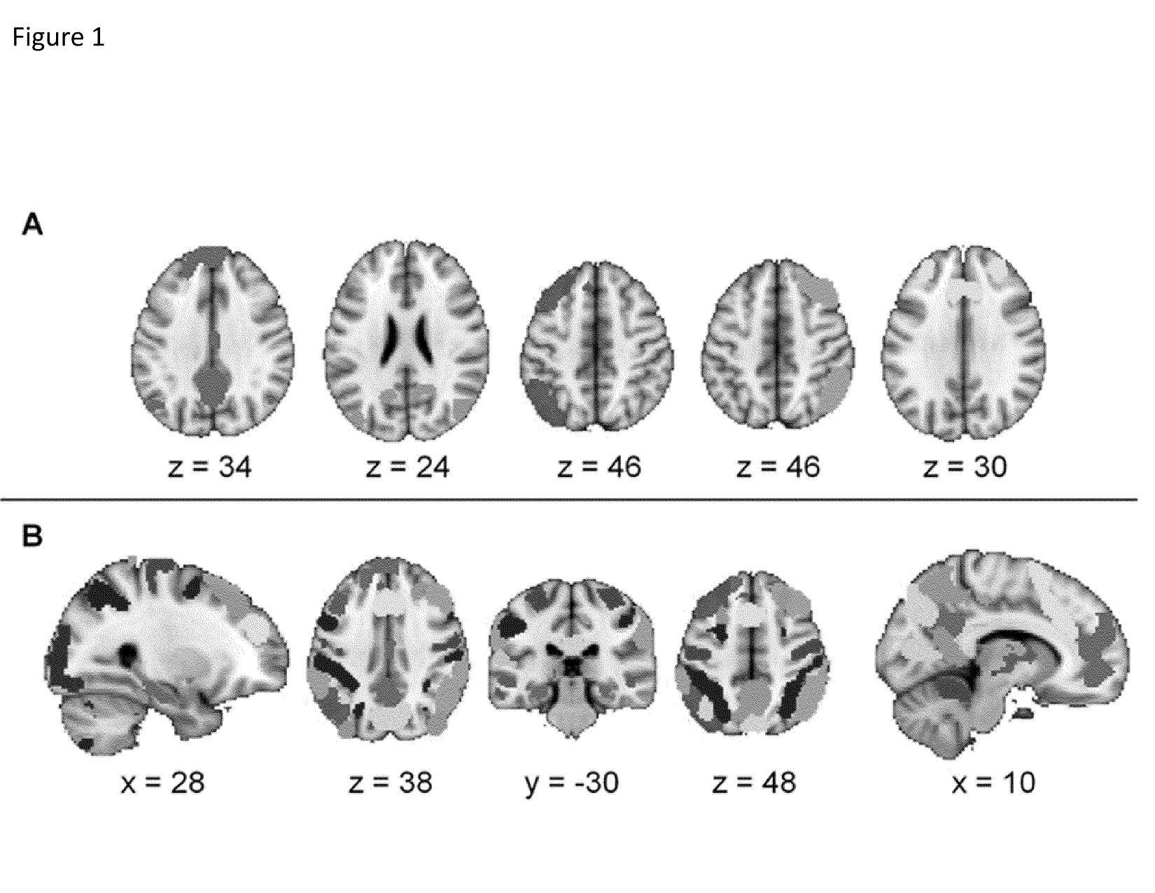

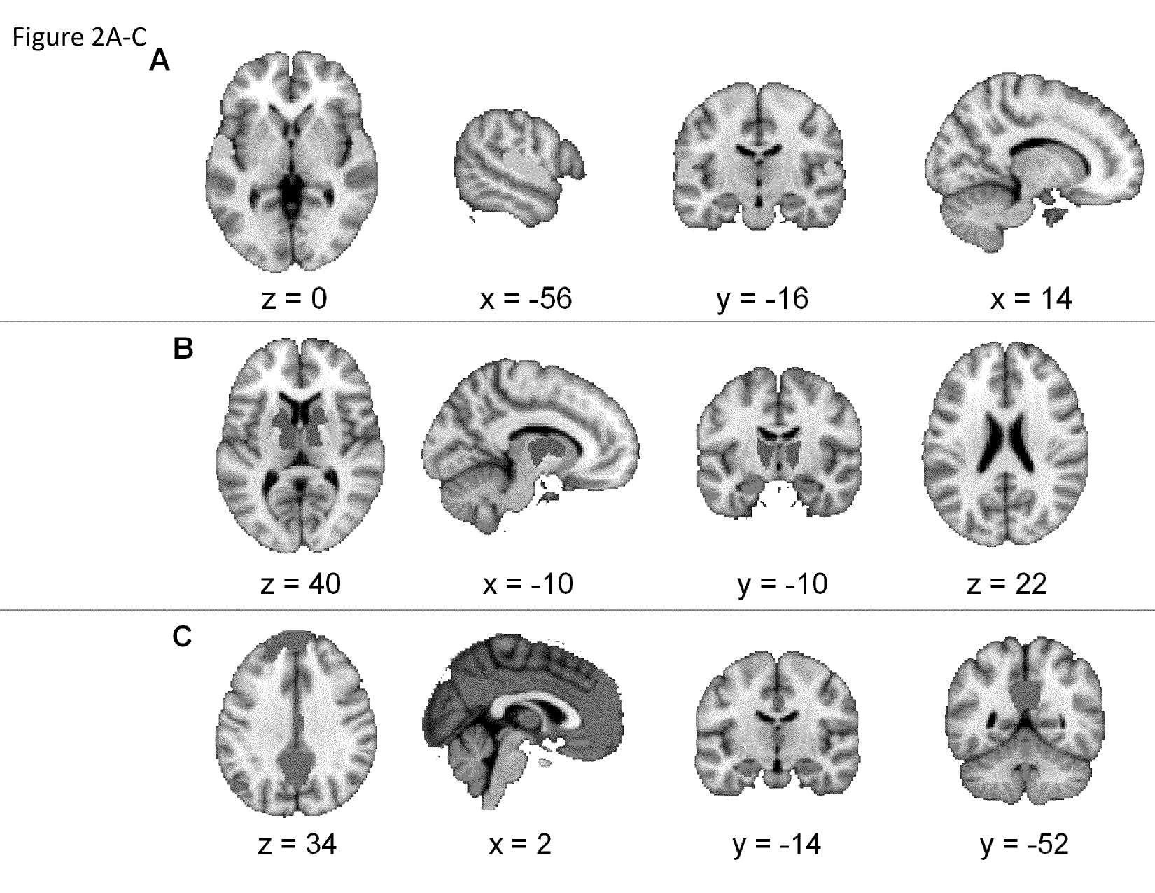

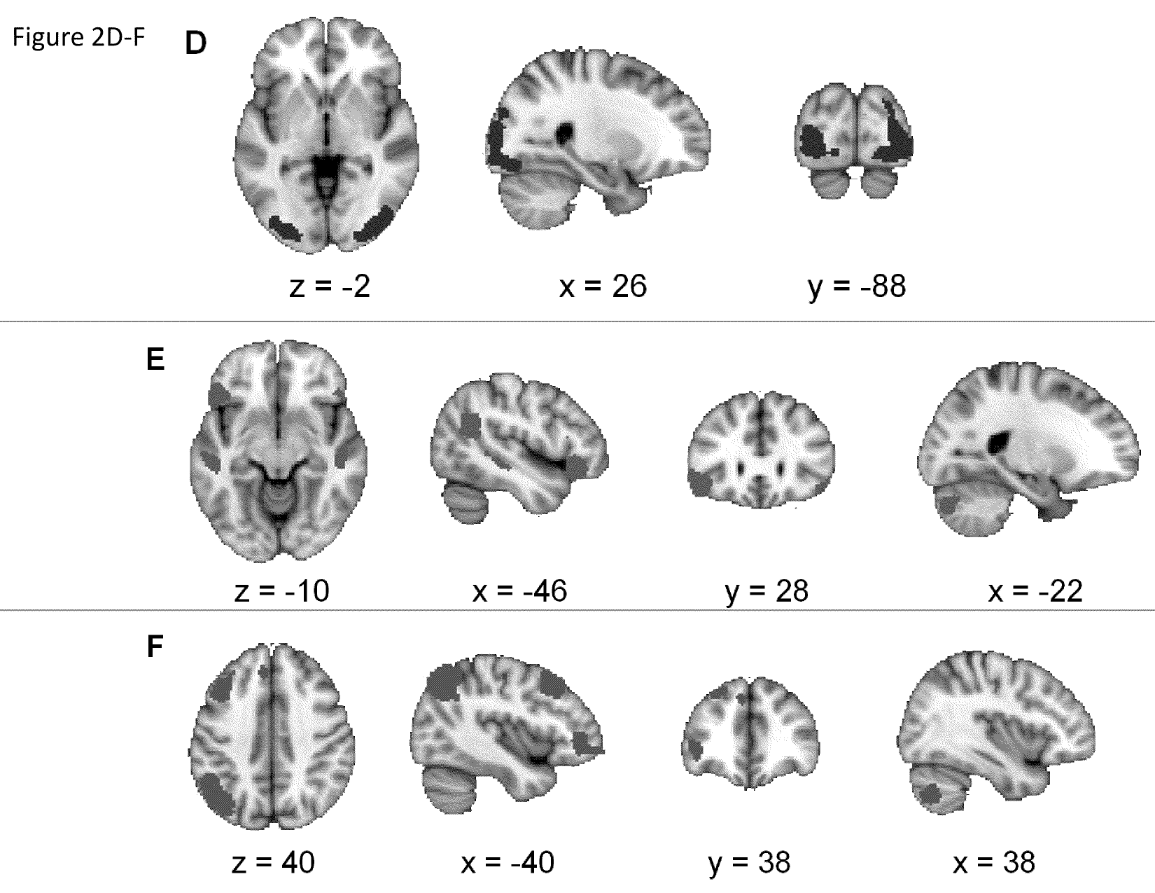

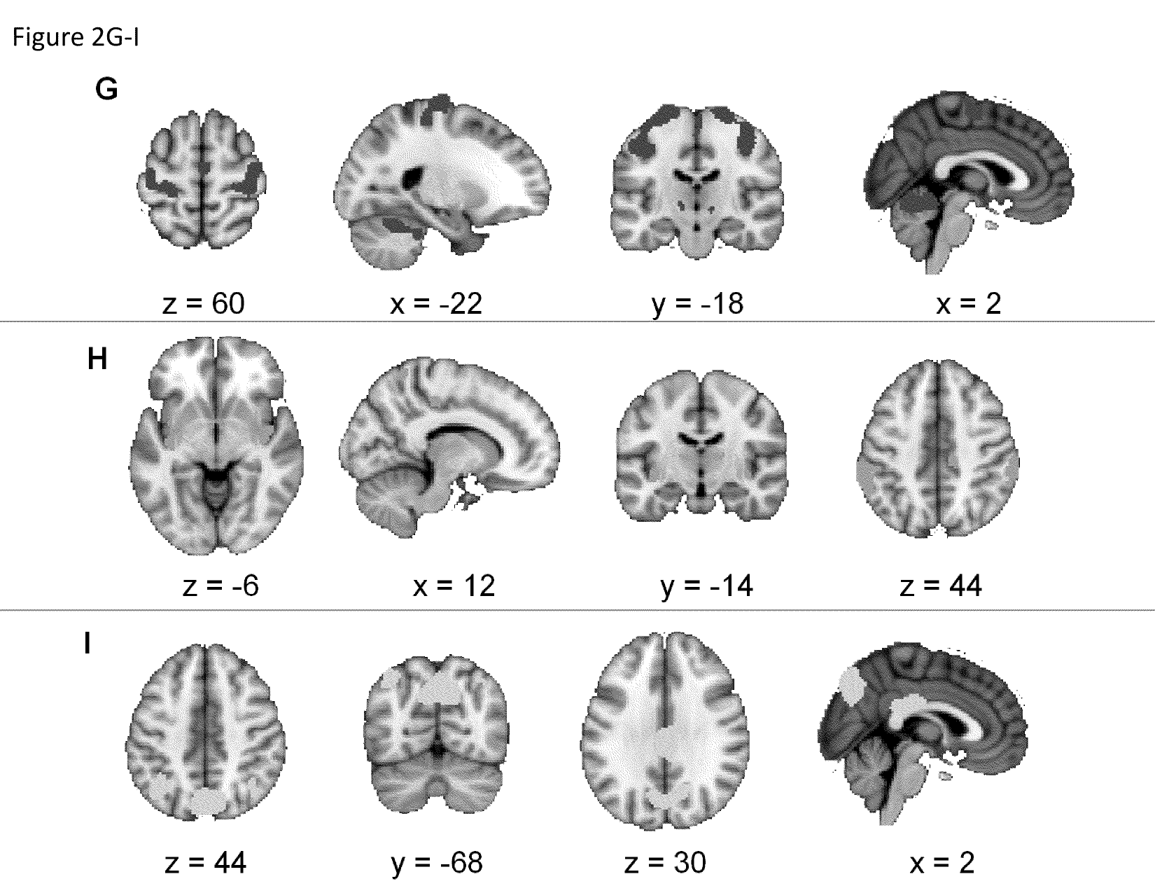

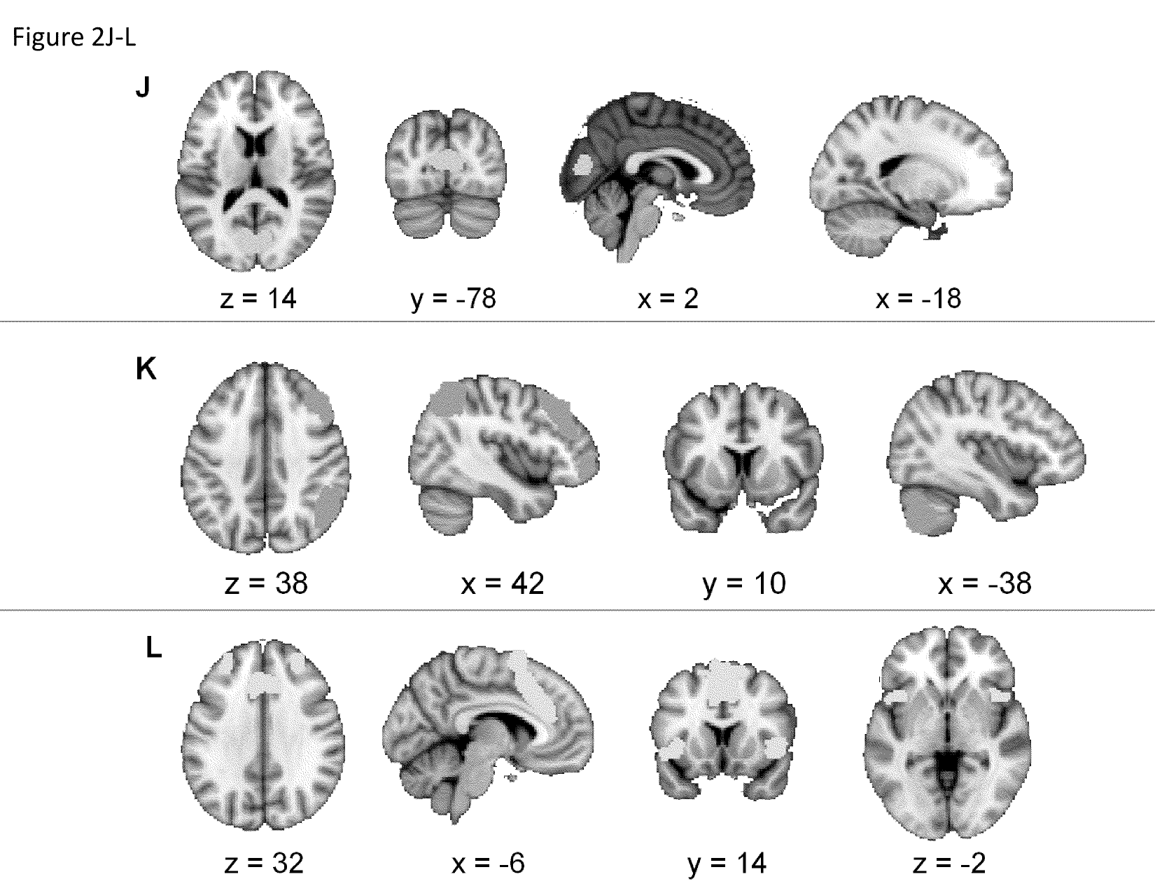

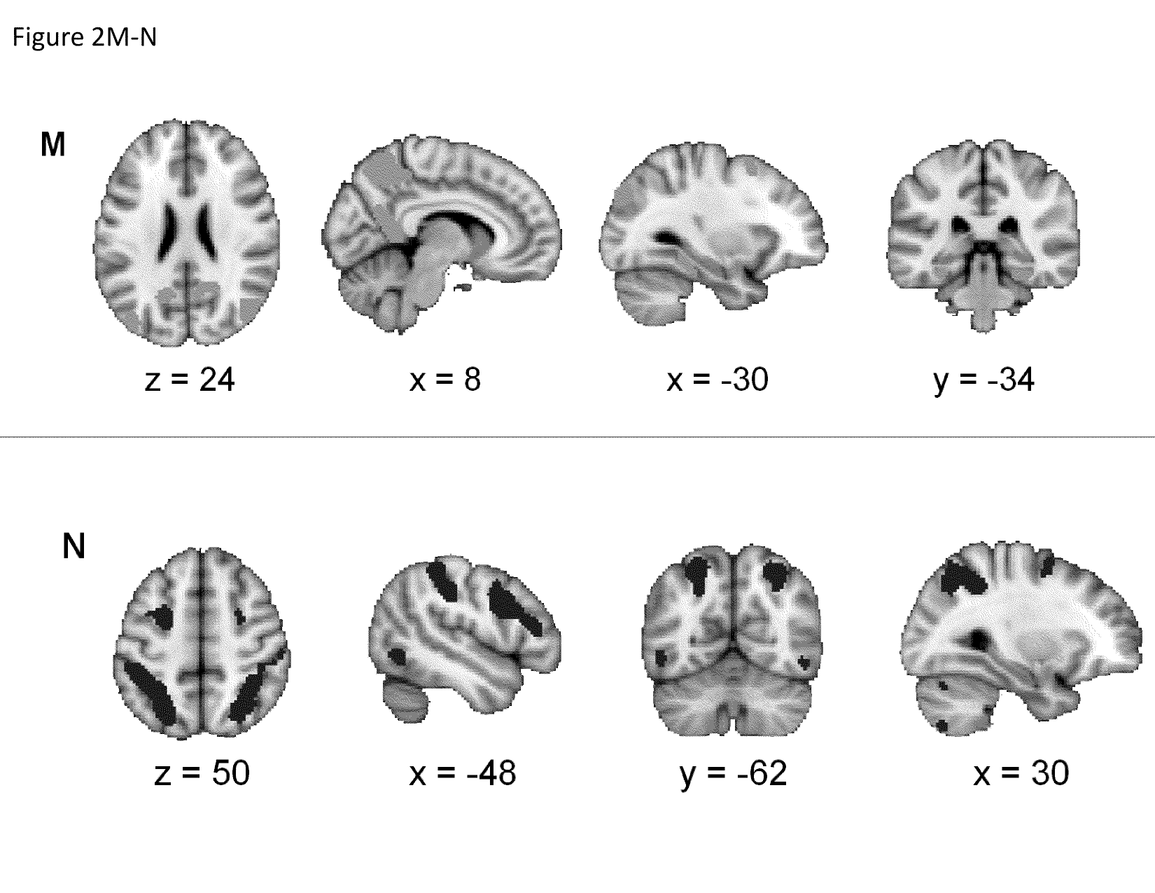

Table 1 describes the anatomical location and Brodmann areas of each of 90 functional ROIs, as detailed in FIGS. 1 and 2.

| | Anatomical Location of | |
|---|---|---|
| | Functional Regions of | Brodmann |
| | Interest (ROIs) | Areas |
| Auditory | Left Superior Temporal Gyrus, | 22, 48 |
| | Heschl's Gyrus | |
| | Right Superior Temporal Gyrus | 22, 38, 42, 48 |
| | Right Thalamus | N/A |
| Basal | Left Thalamus, Caudate | N/A |
| Ganglia | Right Thalamus, Caudate, Putamen | N/A |
| | Left Inferior Frontal Gyrus | 45, 48 |
| | Right Inferior Frontal Gyrus | 45, 48 |
| | Pons | N/A |
| PCC/MPFC | Medial Prefrontal Cortex, | 9, 10, 24, 32, |
| | Anterior Cingulate Cortex, | |
| | Orbitofrontal Cortex | 11 |
| | Left Angular Gyrus | 39 |
| | Right Superior Frontal Gyrus | 9 |
| | Posterior Cingulate Cortex, Precuneus | 23, 30 |
| | Midcingulate Cortex | 23 |
| | Right Angular Gyrus | 39 |
| | Left and Right Thalamus | N/A |
| | Left Hippocampus | 20, 36, 30 |
| | Right Hippocampus | 20, 36, 30 |
| V2 | Left Middle Occipital Gyrus, Superior | 18, 19, 17 |
| | Occipital Gyrus | |
| | Right Middle Occipital Gyrus, | 17, 18, 19 |
| | Superior Occipital Gyrus | |
| Language | Inferior Frontal Gyrus | 45, 47 |
| | Left Middle Temporal Gyrus | 21 |
| | Left Middle Temporal Gyrus, | 21, 37, 39 |
| | Angular Gyrus | |
| | Left Middle Temporal Gyrus, | 21, 22, 42, 40, |
| | Superior Temporal Gyrus, | |
| | Supramarginal Gyrus, Angular Gyrus | 39 |
| | Right Inferior Frontal Gyrus | 47, 45 |
| | Right Supramarginal Gyrus, | 21, 22, 40 |
| | Superior Temporal Gyrus, Middle | |
| | Temporal Gyrus | |
| | Left Crus I | N/A |
| Left DLPFC/ | Left Middle Frontal Gyrus, | 8, 9 |
| Parietal | Superior Frontal Gyrus | |
| | Left Inferior Frontal Gyrus, | 45, 47, 10 |
| | Orbitofrontal Gyrus | |
| | Left Superior Parietal Gyrus, Inferior | 7, 40, 39 |
| | Parietal Gyrus, Precuneus, | |
| | Angular Gyrus | |
| | Left Inferior Temporal Gyrus, | 20, 37 |
| | Middle Temporal Gyrus | |
| | Right Crus I | N/A |
| | Left Thalamus | N/A |
| Sensorimotor | Left Precentral Gyrus, Postcentral Gyrus | 4, 3 |
| | Right Precentral Gyrus, Postcentral Gyrus | 4, 6, 3 |
| | Right Supplementary Motor Area | 6 |
| | Left Thalamus | N/A |
| | Bilateral Lobule IV, Lobule V, Lobule VI | N/A |
| | Right Thalamus | N/A |
| Posterior | Left Middle Frontal Gyrus | 46 |
| Insula | Left Supramarginal Gyrus, | 40 |
| | Inferior Parietal Gyrus | |
| | Left Prenuneus | 5 |
| | Right Midcingulate Cortex | 23 |
| | Right Superior Parietal Gyrus, Precuneus | 7, 5 |
| | Right Supramarginal | 2, 40 |
| | Gyrus, Inferior Parietal Gyrus | |
| | Left Thalamus | N/A |
| | Lobule VI | N/A |
| | Left Posterior Insula, Putamen | 48 |
| | Right Thalamus | N/A |
| | Lobule VI | N/A |
| | Right Posterior Insula | 48 |
| Precuneus | Midcingulate Cortex, | 23 |
| | Posterior Cingulate Cortex | |
| | Precuneus | 7, 19 |
| | Left Angular Gyrus | 7, 40 |
| | Right Angular gyrus | 7, 40 |
| V1 | Calcarine Sulcus | 17 |
| | Left Thalamus | N/A |
| Right | Right Middle Frontal Gyrus, | 46, 8, 9 |
| DLPFC/ | Right Superior Frontal Gyrus | |
| Parietal | Right Middle Frontal Gyrus | 10, 46 |
| | Right Inferior Parietal Gyrus, | 7, 40, 39 |
| | Supramarginal Gyrus, Angular | |
| | Gyrus | |
| | Right Superior Frontal Gyrus | 8 |
| | Left Crus I, Crus II, Lobule VI | N/A |
| | Right Caudate | N/A |
| Insula/dACC | Left Middle Frontal Gyrus | 9, 46 |
| | Left Insula | 48, 47 |
| | Anterior Cingulate Cortex, | 24, 32, 8, 6 |
| | Medial Prefrontal Cortex, | |
| | Supplementary Motor Area | |
| | Right Middle Frontal Gyrus | 46, 9 |
| | Right Insula | 48, 47 |
| | Left Lobule VI, Crus I | N/A |
| | Right Lobule VI, Crus I | N/A |
| RSC/MTL | Left Retrosplenial Cortex, | 29, 30, 23 |
| | Posterior Cingulate Cortex | |
| | Left Middle Frontal Gyrus | 8, 6 |
| | Left Parahippocampal Gyrus | 37, 20 |
| | Left Middle Occipital Gyrus | 19, 39 |
| | Right Retrosplenial Cortex, | 30, 23 |
| | Posterior Cingulate Cortex | |
| | Precuneus | 7, 5 |
| | Right Superior Frontal Gyrus, | 9, 8 |
| | Middle Frontal Gyrus | |
| | Right Parahippocampal Gyrus | 37, 30 |
| | Right Angular Gyrus, Middle | 39, 19 |
| | Occipital Gyrus | |
| | Right Lobule IX | N/A |
| IPS/FEF | Left Middle Frontal Gyrus, | 6 |
| | Superior Frontal Gyrus, Precentral Gyrus | |
| | Left Inferior Parietal Sulcus | 2, 40, 7 |
| | Left Frontal Operculum, | 44, 48, 45 |
| | Inferior Frontal Gyrus | |
| | Left Inferior Temporal Gyrus | 37 |
| | Right Middle Frontal Gyrus | 6 |
| | Right Inferior Parietal Lobule | 2, 40, 7 |
| | Right Frontal Operculum, Inferior | 44, 48 |
| | Frontal Gyrus | |
| | Right Middle Temporal Gyrus | 37 |
| | Left Lobule VIII, Lobule VIIb | N/A |
| | Right Lobule VIII, Lobule VIIb | N/A |
| | Right Lobule VI, Crus I | N/A |

### DEFINITIONS

The practice of the present invention may employ conventional techniques of neurochemistry, neurobiology, cognitive neuroscience, biochemistry and statistics, which are within the capabilities of a person of ordinary skill in the art. Such techniques are fully explained in the literature. For definitions, terms of art and standard methods known in the art, see, for example, Michael S. Gazzaniga ‘The cognitive Neurosciences’, 4^{th} edition, MIT Press 2009; Wilson & Walker ‘Principles and Techniques of Practical Biochemistry’, Cambridge University Press (2000), and S. Kotsiantis ‘Supervised Machine Learning: A Review of Classification Techniques’, Informatic J. 2007, 31:249-268. Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable.

The term “thresholded”, as used herein, relates to image segmentation, whereby digital images from the brain, obtained through, e.g., fMRI, are partitioned into several segments or sets of pixels based on a statistical threshold.

The term “significant” is used herein as a statistical term and refers to a statistical significance level (p-value) of at least 0.05.

The term “subject”, as used herein, refers to a member of a species of mammalian origin.

The terms “rest state” and “resting state”, as used herein, refer to a state where a subject is not carrying out any specific task.

The term “memory state”, as used herein, refers to a state where a subject carries out a complex cognitive task.

The term “difference matrix”, as used herein, defines the matrix that remains after a subject's rest state matrix is subtracted from the same subject's memory state matrix.

The term “disease progression”, as used herein, defines a specific pattern of pair-wise correlation coefficients between defined regions of interest (ROIs) in the brain that becomes progressively more distinct from a healthy control pattern.

The term “treatment success”, as used herein, defines a specific pattern of pair-wise correlation coefficients between defined regions of interest (ROIs) in the brain that becomes progressively closer to a healthy control pattern.

The term “intrinsic connectivity networks (ICNs)”, as used herein, refers to a host of resting state brain networks with distinct spatial and temporal profiles corresponding to canonical functions such as vision, hearing, language, working memory, visuospatial attention, salience processing, and episodic memory.

Independent component analysis. Independent component analysis (ICA) is a statistical technique that separates a set of signals, for example fMRI data, into independent—uncorrelated and non-Gaussian—spatiotemporal components. Functionally connected networks were identified through group spatial independent component analysis of fMRI data by estimating spatially independent patterns from their linearly mixed fMRI signals. In order to test for functional brain connectivity, the structure and function of those complex, functionally connected neuronal networks were analyzed by temporally correlating localized activity in the brain. Two regions of interest correlate positively, if changes in activity over time are correlated across the two. If changes in activity observed in the one region of interest are inversely correlated with changes in activity in the second region, then two regions of interest correlate negatively.

Pattern recognition analysis and data output. In order to identify and classify specific cognitive states or traits, as indicated by specific patterns of pair-wise correlation coefficients between defined regions of interest (ROIs), from brain imaging data, statistical tests for pattern recognition were employed to identify whole-brain functional connectivity markers. Following data analysis, the identified whole-brain functional connectivity markers are transformed into information for graphical display or output to a computer-readable medium, computer or computer network.

### DETAILED DESCRIPTION

Specific cognitive traits and states can be distinguished and classified according to unique patterns of activity in a network of coordinated and mutually communicating brain regions. Neuropsychiatric diseases and disorders can disrupt these networks and cause specific variations in the whole-brain connectivity profile, which can be used for diagnostic testing, monitoring of disease progression or treatment success.

The present invention provides for improved brain imaging and decoding methods that test subjects under authentic, natural conditions that allow for regular patterns of free-flowing thought and perception, as they occur in everyday life, while taking into account brain activities that were measured over spatially diverse regions of the whole brain (whole-brain connectivity signatures). From such whole-brain connectivity signatures, specific cognitive traits and states are decoded and classified in a whole-brain connectivity analysis which takes into account the full pattern of brain activity.

The determination of specific cognitive traits in neurotypical subjects, who represent healthy control subjects with a neurotypical profile, in comparison to specific cognitive traits in neuro-atypical subjects, who deviate from a neurotypical profile in some form, can provide important guidance in the clinical diagnosis of neuropsychiatric diseases and disorders, in the monitoring of neuropsychiatric disease progression and in the monitoring of neuropsychiatric treatment success.

In the nonclinical fields of neuromarketing and neuroeconomics, the determination of specific cognitive states can aid in predicting consumer behavior and financial decision making. So can the determination of specific cognitive states in subjects who are offered a product for sale at a particular condition, e.g. at a particular price, in comparison to specific cognitive states in control subjects who are not offered a product can indicate a subject's perception and reaction to an offered product or price for a product. Such indicators can be instrumental in guiding product offering and product pricing.

The Use of Functional Connectivity Magnetic Resonance Imaging (Functional Connectivity MRI), in Contrast to Standard fMRI, in Obtaining Whole-brain Connectivity Signatures

Standard functional magnetic resonance imaging (fMRI) is an imaging technique with high spatial resolution that not only provides the ability to detect and map activated structures in several dimensions inside the body, particularly inside the brain, but also the ability to image which internal structures, even spatially remote ones, contribute to certain functions by imaging changes in brain hemodynamics (blood flow and oxygen consymption) that correspond to neuronal activity. For high spatial resolution, three dimensions (x, y, z-axes) are imaged and a magnetic field is applied perpendicular to a desired plane.

In standard fMRI studies cognitive subtraction experiments measure blood-oxygen level-dependent (BOLD) signal changes across two or more states (usually under resting state conditions which serve as the control conditions and under testing conditions), and the precise start and stop times of each state are required. A major obstacle to decoding subject-driven cognitive traits states has been the functional imaging field's reliance on such cognitive subtraction experiments (Friston, 1998). By contrast, functional connectivity MRI, as used herein, examines BOLD signal correlations across brain regions and can be performed over single free-streaming states.

Analysis of task-activation fMRI data has been used for decoding brain states in such a carefully controlled experimental setting. However, the need to switch between experimental and control conditions and the need to control stimulus timing impede the use of task-activation fMRI to study naturalistic brain processes which are typically continuous (rather than discontinuous) and subject-driven (rather than investigator-driven).

Resting-state fMRI is a distinct approach that examines functional connectivity between different brain regions while a subject rests quietly in the scanner. This technique commonly involves examining connectivity within networks of roughly 6-10 brain regions. Changes in resting-state connectivity profiles within particular brain networks have been used to classify subjects into diagnostic groups, for example, Alzheimer's disease versus frontotemporal dementia or healthy aging (Greicius et al., 2004; Zhou et al., 2010). One study examined functional connections between 90 structurally-defined regions-of-interest (ROIs) and used a global measure of connectivity strength to classify subjects suffering from Alzheimer's disease from control subjects (Supekar et al., 2008).

Functional Regions of Interest Versus Structural Regions of Interest

In one aspect of the present invention, a subject's brain was carefully parcelled into a large number (90+) of functional regions of interest (ROIs) to provide a vast functional connectivity matrix, where distinct cognitive states and traits can be isolated and defined in a full exploration of the entire brain. If, as demonstrated in one embodiment of the invention, a subject's brain is parceled into 90 ROIs, a matrix of 3,960 pairwise correlations is produced, whose specific patterns indicate a distinct cognitive state or condition. If a subject's brain is parceled into 100+ ROIs, a matrix of 5,000+ pairwise correlations is produced, and so forth.

The use of functionally defined ROIs instead of purely structurally defined ROIs enables a far more accurate assessment of functional brain connectivity. In embodiments of the present invention, classification with functional ROIs proved to be more accurate than classification with structural ROIs, as described in Example 2. In comparison to functional ROIs, structural ROIs are much more coarsely defined and often encompass and combine several functionally distinct regions which carries the risk that meaningful information from those brain regions is diluted or lost and that, as a consequence, the classification potential is weakened. Furthermore, combining two or more functional ROIs into a single structural ROI has the potential to introduce errors by creating novel, hybrid structural ROI time series that do not reflect the true functional information of either functional ROI, but, instead, result in an aggregated and incorrect functional signal.

Specific Pattern Analysis of Whole-brain Connectivity Using Functional Regions of Interest in a Functional Connectivity Matrix

In embodiments of the present invention, functional connectivity MRI data were used to define functional regions of interest (ROI) for the entire brain and also to train a classifier for classification with the objective to identify and classify specific patterns between regions of interest that can serve as reliable markers of a specific, cognitive state or a general cognitive trait.

As described in various embodiments of the invention, specific pattern analysis of whole-brain connectivity was used to distinguish between four subject-driven cognitive states, namely undirected rest, retrieval of recent episodic memories, serial subtractions, and (silent) singing of music lyrics. To achieve this, ninety functional regions-of-interest (ROIs) were defined across 14 large-scale resting-state brain networks to generate a 3960 cell matrix reflecting whole-brain connectivity. In such a vast functional connectivity matrix temporally correlating brain regions can be described, regardless of their spatial proximity or remoteness, and distinct cognitive states and traits can be isolated and defined based on their specific whole-brain functional connectivity signatures.

Classification algorithm. To identify specific patterns of whole-brain connectivity in embodiments of the present invention, a classifier was trained, as subjects rested quietly, remembered the events of their day, subtracted numbers, or (silently) sang lyrics. In a leave-one-out cross-validation the classifier identified these four cognitive states with 84% accuracy. More critically, the classifier achieved 85% accuracy when identifying these states in a second, independent cohort of subjects. Classification accuracy remained high with imaging runs as short as 30-60 seconds. At all temporal intervals assessed, the 90 functionally-defined ROIs outperformed a set of 112 commonly-used structural ROIs in classifying cognitive states. The generalizability of the classification algorithm was tested with two methods: leave-one-out cross-validation (LOOCV) and cross-validation on an independent cohort.

Continuous data acquisition. In embodiments of the present invention, functional connectivity imaging data were acquired in continuous ten-minute runs with no stimulus presentation and no investigator-imposed temporal landmarks other than the start and end of the scan. Significantly shorter scans (lasting several seconds long) and longer scans are contemplated as well.

Neuropsychiatric Diseases

Neuropsychiatric diseases, in particular, age-related disorders such as neurodegenerative diseases are becoming an increasing social and economical burden as the number of older individuals continues to grow in industrialized countries. Examples of neuropsychiatric diseases that do not involve neurodegeneration include but are not limited to chronic pain, depression, anxiety, etc.

Neuropsychiatric Diseases Involving Neurodegeneration

Alzheimer's disease. Alzheimer's disease is a devastating, degenerative disorder of the brain and currently the leading cause as well as most prevalent form of dementia in the elderly; Alzheimer's disease starts phenotypically with memory loss and eventually results in complete loss of intellectual and everyday life skills Despite the progress which has been achieved in elucidating the underlying mechanisms of Alzheimer's disease and related forms of dementia, there remains an urgent need to develop methods for early diagnosis. For example, current diagnosis of milder forms of Alzheimer's disease, where obvious, phenotypical signs of dementia such as loss of orientation or loss of memory are still lacking, cannot reliably and directly be assessed, but has to be performed by exclusion of other neurological disorders (Dubois et al., 2007).

Vascular dementia. Vascular dementia (aka multi-infarct dementia), is currently the second most prevalent form of dementia in the elderly and is characterized by vascular lesions in the brain. Early detection and diagnosis are important, since vascular dementia can at least partially be prevented, when diagnosed early enough.

Parkinson's disease. Parkinson's disease is a degenerative disorder of the central nervous system that affects motor skills, speech and also cognitive functions and is characterized by muscle rigidity, tremor and extremely slow physical movements.

Lewy body dementia. Lewy body dementia, a synucleinopathy, is phenotypically closely associated with both Alzheimer's and Parkinson's diseases and is characterized anatomically by the presence of Lewy bodies, which are cytoplasmic inclusions of alpha-synuclein and ubiquitin protein, in neurons.

Frontotemporal dementia. Frontotemporal dementia is believed to be caused by degeneration of the frontal lobe and possibly also of the temporal lobe of the brain, greatly affecting cognitive functions, language skills and behavior.

Prion disease. Prion disease (aka transmissible spongiform encephalopathies) represents a group of neurodegenerative disorders that affect humans and animals alike and that are transmitted by prions or other similar infectious organisms. The disorders cause impairment of brain function, including memory loss, personality changes and impaired physical movement.

Huntington's disease. Huntington's disease is a progressive, neurodegenerative, genetically based disorder that results from brain damage caused by aggregats of misfolded huntingtin protein and that affects muscle coordination and cognitive functions, typically from middle age on.

Tauopathies. Tauopathies are neurodegenerative disorders that result from the toxic aggregation of tau protein in neurofibrillary tangles in the brain.

Neuropsychiatric Diseases not Involving Neurodegeneration

Chronic pain. Pain can be acute or chronic,malignant or nonmalignant, nociceptive or neuropathic. In any case, accurate classification of pain is difficult, since pain perception and tolerance thresholds are different for every subject. A method of classifying various degrees of pain in a subject using whole-brain functional connectivity signatures would be very helpful in the conduction of clinical studies for analgesics to ensure an objective measurement of pain instead of a subjectively judged sensation.

Depression and anxiety disorders. Depression and anxiety disorders can manifest in extremely different ways and degrees of emergencies Like pain classification, an accurate classification of depression and anxiety disorders is difficult; the classification of cognitive traits in subjects with possible depression or anxiety disorders would be beneficial.

Neuromarketing

Neuromarketing defines an area within marketing that studies subjects' cognitive or subcognitive states and conditions in response to an exposure towards certain product-related stimuli. Neuromarketing has a particular interest in identifying the particular brain areas that are activated in response to an exposure to such product-related stimuli as to uncover the real desires and needs of consumers.

Neuroeconomics

Neuroeconomics defines an overlapping area of neurosciences and economics and that studies subjects' cognitive states, conscious or subconscious, and conditions in situations of financial investments and financial decisions to uncover the underlying motives and reasons for certain financial decision making.

Evaluation of Whole-brain Functional Connectivity Signatures to Classify Cognitive States and Traits

Following the acquisition of brain images of a subject via functional MRI in resting state as well as in one or more non-resting (task) states, a resting state matrix as well as “non-resting” or “task-driven” matrices are produced by calculating pairwise correlations between all (90+) regions of interest identified. There are several possible approaches to identifying a state-specific matrix. One can subtract the resting state matrix from a non-resting state matrix to obtain a difference matrix which distinguishes rest from the cognitive state. Alternatively, one can examine the functional connectivity matrices of several states (rest, memory retrieval, visuospatial attention, emotional processing, watching a movie, etc . . . ) and determine what features of the matrices are unique to a specific state. This provides a state-specific pattern of whole-brain functional connectivity. These state-specific connectivity matrices can be defined across a group of subjects showing correlations that are both state-specific and consistent across subjects. These group-level state matrices can then be used to classify subsequent, independent individual connectivity matrices as reflecting rest, memory processing, visuospatial attention, etc. Similarly group-level state matrices can be defined for specific neuropsychiatric disorders such as Alzheimer's disease or Parkinson's disease. These group-level matrices can then be used to classify single-subjects based on how well their single-subject whole-brain connectivity matrix matches a disease group matrix.

In a physical transformation step, this result can be graphically displayed, for example, on a computer screen. Furthermore, this result can be outputted to a computer readable medium.

Utility of the Present Invention

The ability to decode and distinguish specific cognitive states or traits from brain imaging data, e.g., through functional magnetic resonance imaging, constitutes a major goal in cognitive neuroscience for many reasons. For example, the assessment and classification of complex cognitive traits such as Alzheimer's disease or depression from resting-state brain connectivity patterns would constitute a valuable diagnostic tool. Alzheimer's disease is a highly prevalent neurodegenerative condition in the industrialized world and, to date, cannot be diagnosed with complete certainty until after the death of an afflicted subject. The assessment of such an afflicted subject's stable cognitive trait might considerably aid in properly diagnosing a subject who is afflicted with Alzheimer's disease and, furthermore, to stratify that subject into, e.g., mild, moderate or advanced to provide the treating physician with the key information needed to select the most effective and most suitable treatment option for the assessed stage of neurodegenerative disease. Furthermore, the effectiveness of a neuropsychiatric treatment regimen in subjects suffering from a neuropsychiatric disease or disorder can be evaluated and monitored using whole-brain functional connectivity analysis, for example, by analysis before and after treatment or by analysis on treatment versus off treatment. Moreover, a quick assessment of whole-brain connectivity signatures might serve as a rapidly available clinical diagnostic marker in the emergency room setting to discern newly checked-in subjects who just suffered a stroke and urgently require proper treatment from subjects who did not experience a stroke, but suffer from a possibly still undiagnosed or undisclosed neuropsychiatric disease or disorder.

In addition to being used to define stable cognitive traits related to distinct neuropsychiatric disorders, the methods described here can be used to define patterns of connectivity that reflect specific cognitive states. Defining, for example, the connectivity pattern that reflects emotional processing or emotional engagement would allow one to assess whether a subject viewing an advertisement or movie clip was emotionally engaged.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

Experimental Procedures

The following methods and materials were used in the examples that are described further below.

Subjects and Tasks

Twenty-seven healthy right-handed subjects (10 males and 17 females) aged 18 to 30 participated in this study. These subjects were recruited in two cohorts of 15 and 12 subjects separated by a 5-6 months interval and were treated as independent cohorts for training and validating the classifier. Data from the first 15 subjects ('classifier cohort') were used to define the functional regions of interest (ROIs). Data from 14 of the same 15 subjects were used to train the classifier (one subject was excluded due to unusable data from the memory scan). Data for the 12 remaining subjects (‘validation cohort’) were collected five months later. Ten of these subjects' data were used to test the classifier; two subjects from the second cohort were excluded for falling asleep. The experimental protocol was approved by the Institutional Review Board of Stanford University.

The classifier cohort of subjects completed four ten-minute tasks: a resting-state scan (also referred to as rest scan or rest task), an episodic memory task, a music lyrics task, and a subtraction task. The rest scan was always completed first, and the order of the three cognitive tasks was counterbalanced. For the rest task, subjects were instructed to close their eyes, let their minds wander, and try not to focus on any one thing. For the memory task, subjects were asked to recall the events of the day from when they awoke until they lay down in the scanner. For the music task, subjects were asked to sing their favorite songs in their head. For the subtraction task, subjects were asked to count backwards from 5000 by 3s. Subjects were instructed to keep their eyes closed during each of the self-driven cognitive states. The 12 subjects from the validation cohort completed an additional task, in which they were asked to imagine walking through all the rooms of their house or apartment (Owen et al., 2006). Debriefing of subjects confirmed that all but two were awake throughout the scans and were able to perform the self-driven tasks for the entire 10 minutes.

Imaging Methods and Data Processing

Functional Magnetic Resonance (fMRI) Acquisition. Functional images were acquired on a 3 Tesla General Electric scanner using an 8-channel head coil. To reduce blurring and signal loss arising from field inhomogeneities, an automated high-order shimming method based on spiral acquisitions was used (Kim et al., 2002). Thirty-one axial slices (4 mm thick, 0.5 mm skip) covering the whole brain were imaged using a T2* weighted gradient echo spiral pulse sequence (TR=2000 msec, TE=30 msec, flip angle=80° and 1 interleave) (Glover & Lai, 1998; Glover & Law, 2001). The field of view was 220×220 mm², and the matrix size was 64×64, giving an in-plane spatial resolution of 3.4375 mm.

fMRI Analysis. Data were preprocessed and analyzed using SPM5 (www.fil.ion.ucl.ac.uk/spm). Images were corrected for movement using least square minimization and normalized to the Montreal Neurological Institute template (Friston et al., 1995). Images were then resampled every 2 mm using sinc interpolation and smoothed with a 6mm Gaussian kernel. Resampling and smoothing were done in 3 dimensions yielding a 2 mm³ resolution and effective spatial smoothness (full width at half maximum) of 7.2×7.1×8.4 mm. The difference in the x and y dimensions reflects imprecision in the measurement as calculated by SPM's smoothness algorithm. A high-pass filter was applied to remove low-frequency signals (<0.008 Hz) from the data. A low pass filter is often used in resting-state analyses, but was excluded here to retain potentially useful information in the higher frequency bands, particularly during the cognitive tasks. To test the utility of high frequency data in classifying, an analysis using a bandpass filter, which filtered out the high frequency data, was included, which resulted in a significantly reduced classification accuracy (see FIG. 2). It is worth noting that cardiac and respiratory signals are known to cause noise in high frequency bands. To account for such possible interference, the subjects' heart rate and respiration rate were measured while they were being scanned. These data were used to regress the participants' physiological noise from their fMRI data (Chang and Glover, 2009).

Classifier Development

Creation of regions of interest (ROI). Regions of interest (ROIs) were created by applying FSL's group melodic independent component analysis (ICA) software (http://www.fmrib.ox.ac.uk/fsl/melodic/index.html) to the group-level resting state data for the first 15 subjects. Of the 30 components generated, 14 were selected visually as being intrinsic connectivity networks (ICNs) based on previous reports by the inventors and others (Damoiseaux et al., 2006; Fox et al., 2005; Greicius et al., 2003; Kiviniemi et al., 2009; Seeley et al., 2007; Smith et al., 2009). Each of the 14 ICNs was thresholded independently and arbitrarily to generate distinct, moderately sized ROIs in the cortex and subcortical gray matter (z=7.0±0.47; zₘₐₓ=9; zₘᵢₙ=4.6; voxels≧25). The subcortical clusters in most ICNS are less robust and a lower threshold was used to isolate these (z=3.8±0.40; zₘₐₓ=5; zₘᵢₙ=2.5; voxels≧15). This thresholding step resulted in 90 ROIs across the 14 ICNs covering most of the brain's gray matter (FIGS. 1 and 2). ROI generation was done prior to classification training and was not driven by classification results. These 90 ROIs are available for download from the inventors' website (http://findlab.stanford.edu).

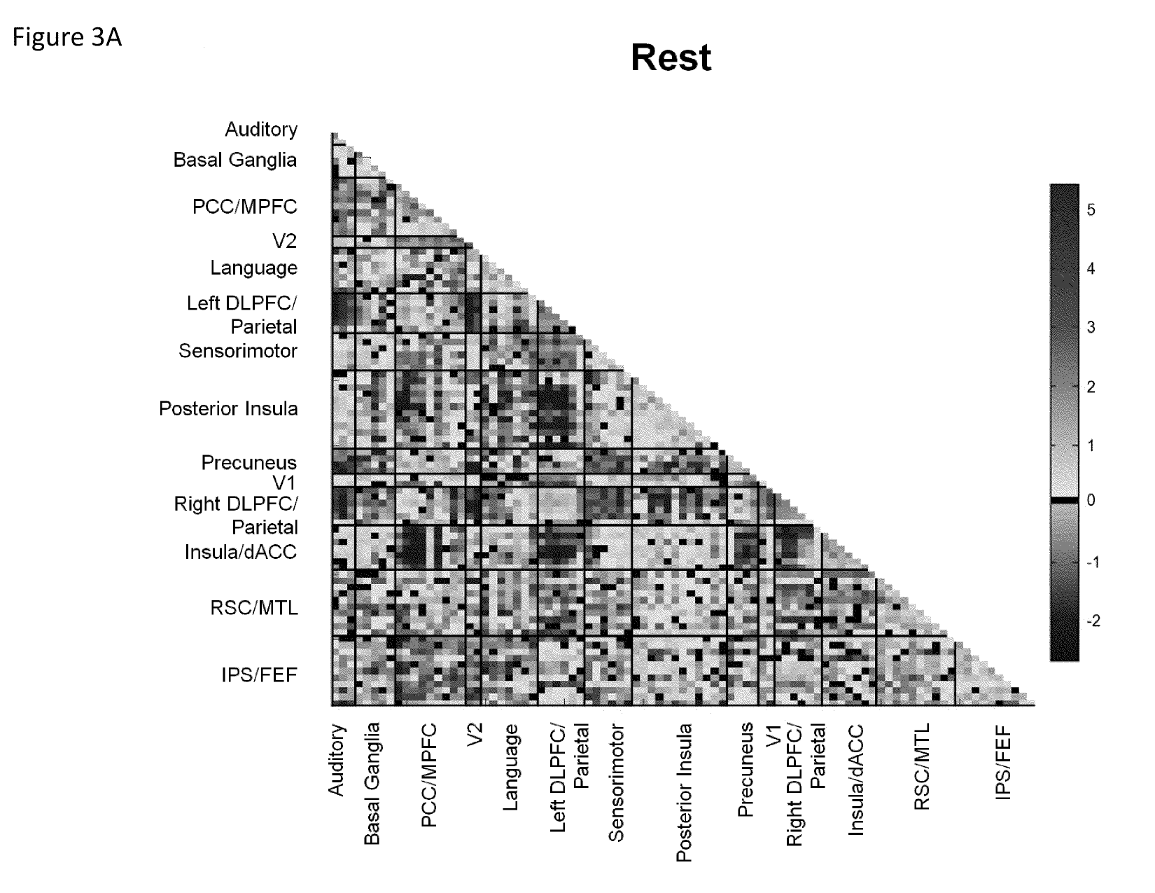

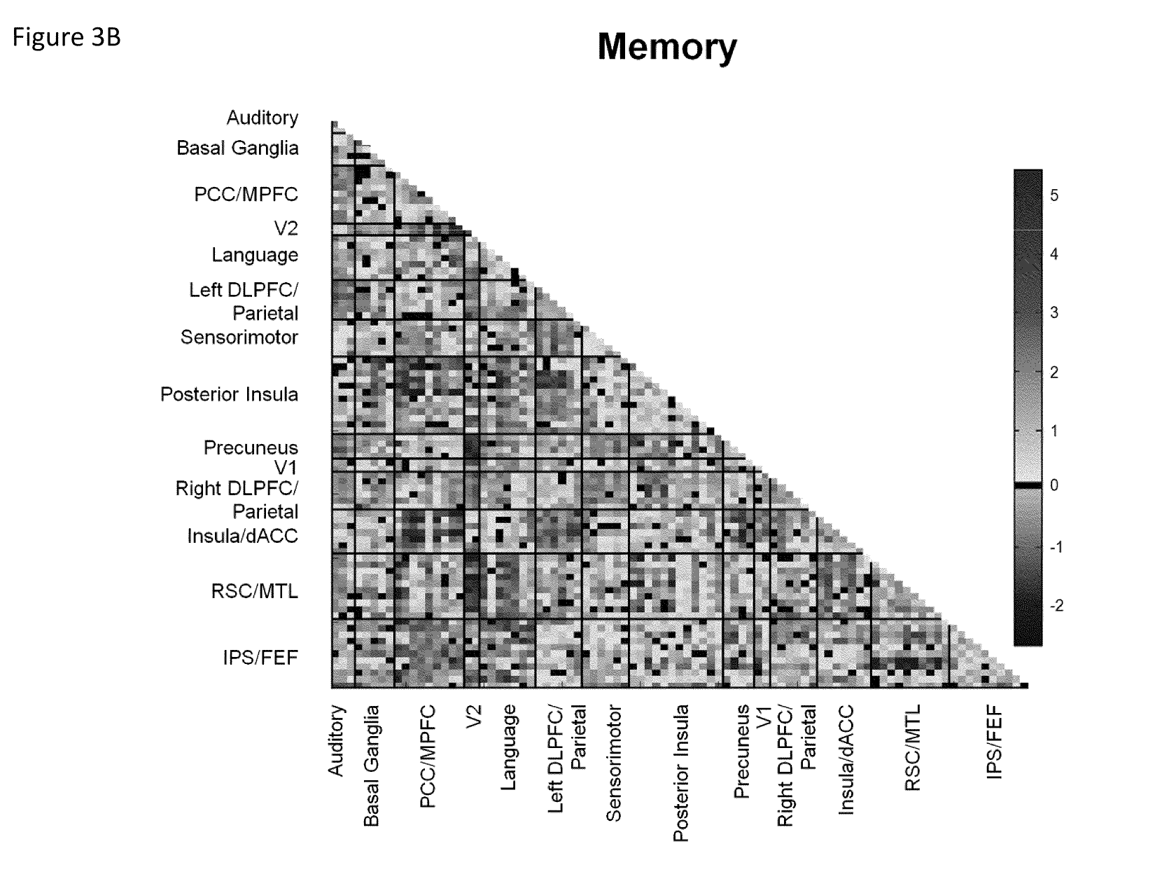

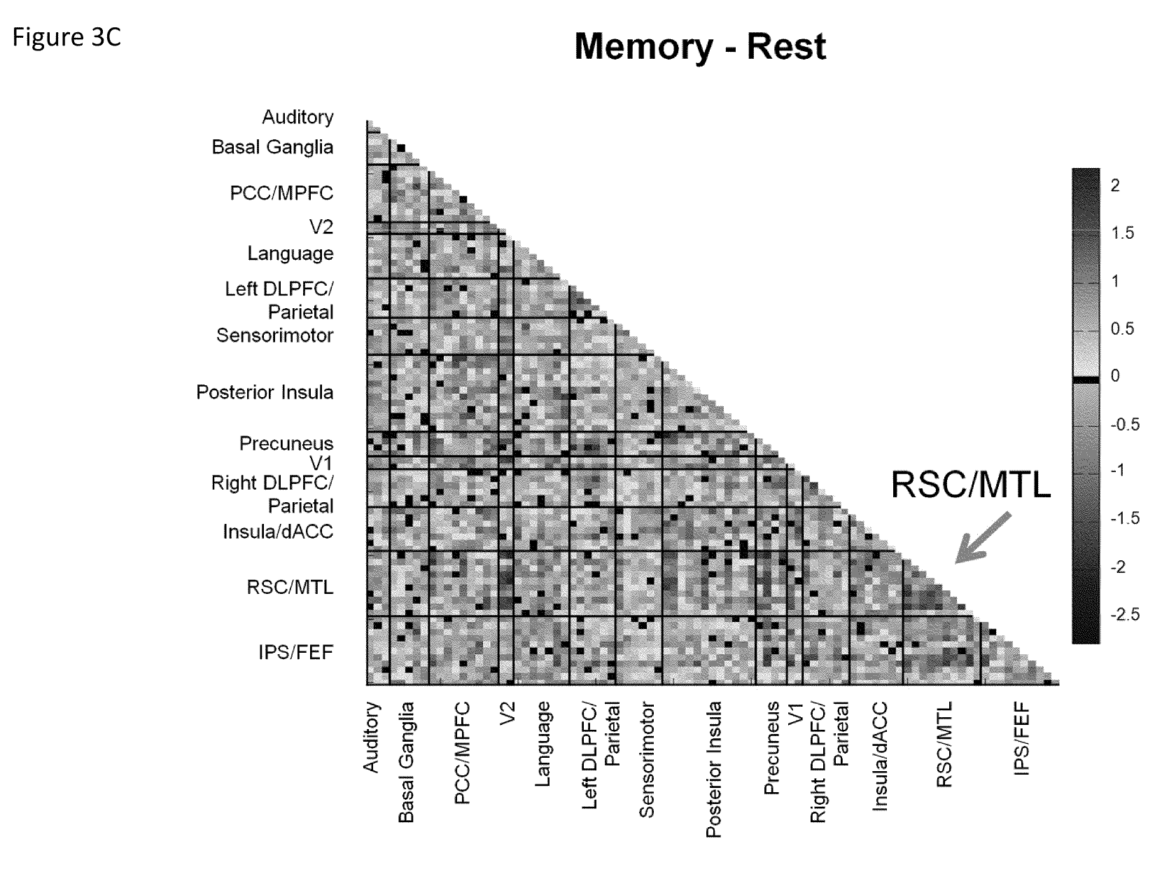

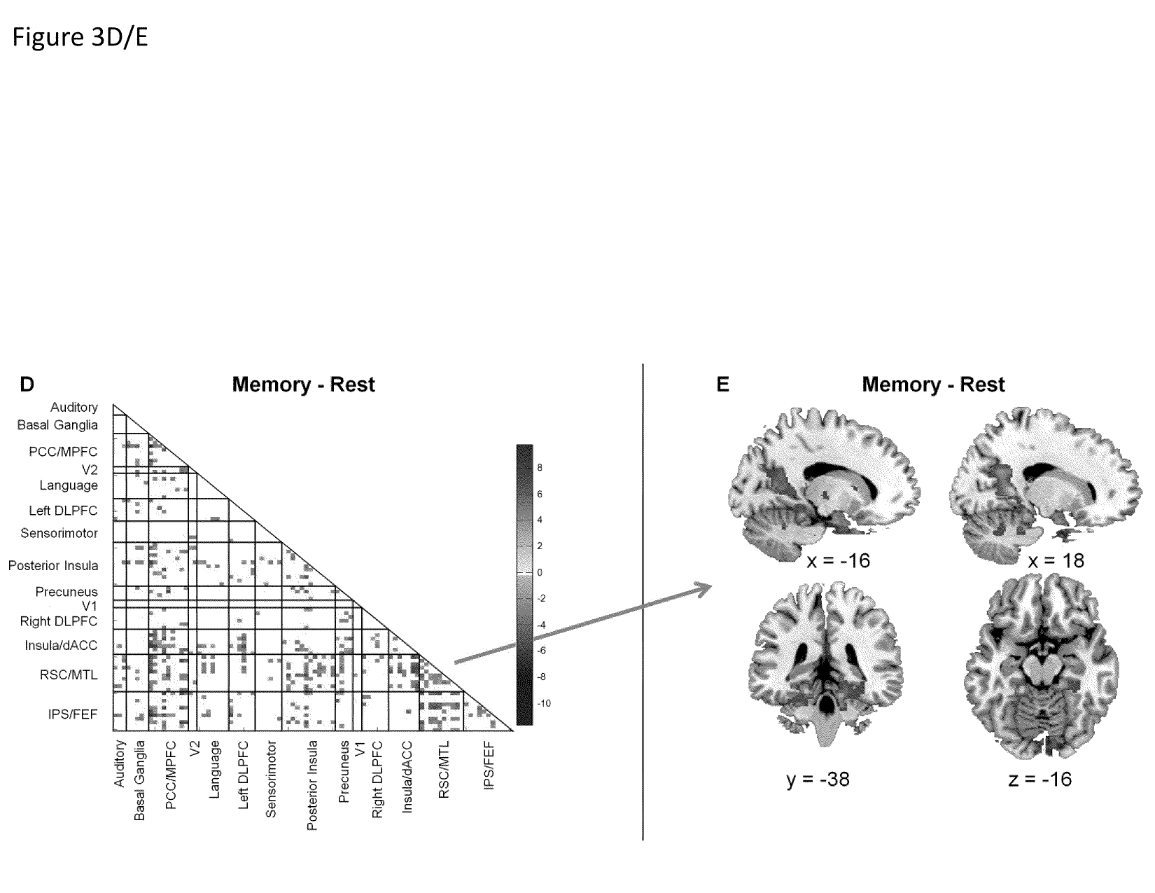

Individual subject functional connectivity matrices. Fourteen subjects from the classifier cohort had usable data in the resting-state scan and the three additional subject-driven cognitive tasks: memory, subtraction, and music. The functional connectivity (FC) between the 90 ROIs was measured during rest and the three different cognitive tasks (see FIG. 3). For each ROI time series the global mean and the confounding effects of CSF and white matter were regressed out. The pearson correlation coefficient was then calculated between the time series of all ROIs, and these correlation coefficients were then converted to z-scores by applying the Fisher transformation. This resulted in an 3960 cell matrix of FC for each of the four cognitive states in every subject. Individual subject functional connectivity matrices were created in the same manner for the spatial navigation task in the validation cohort.

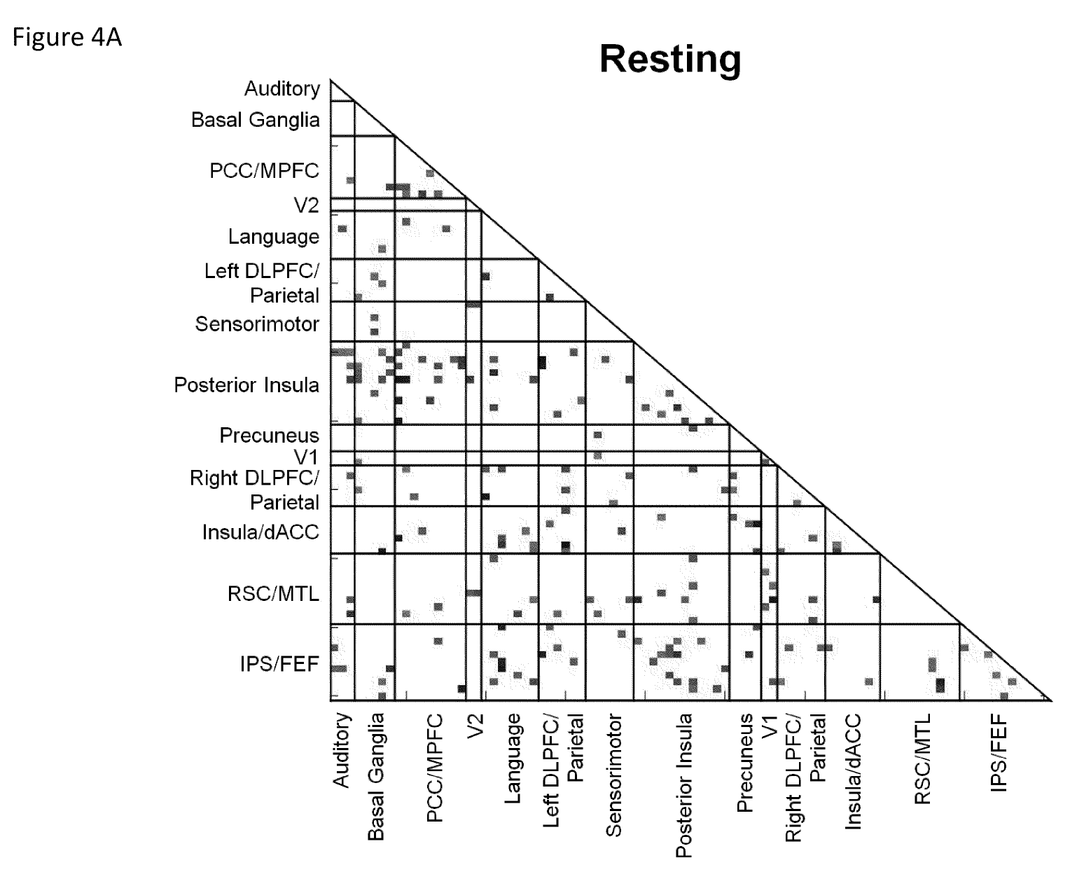

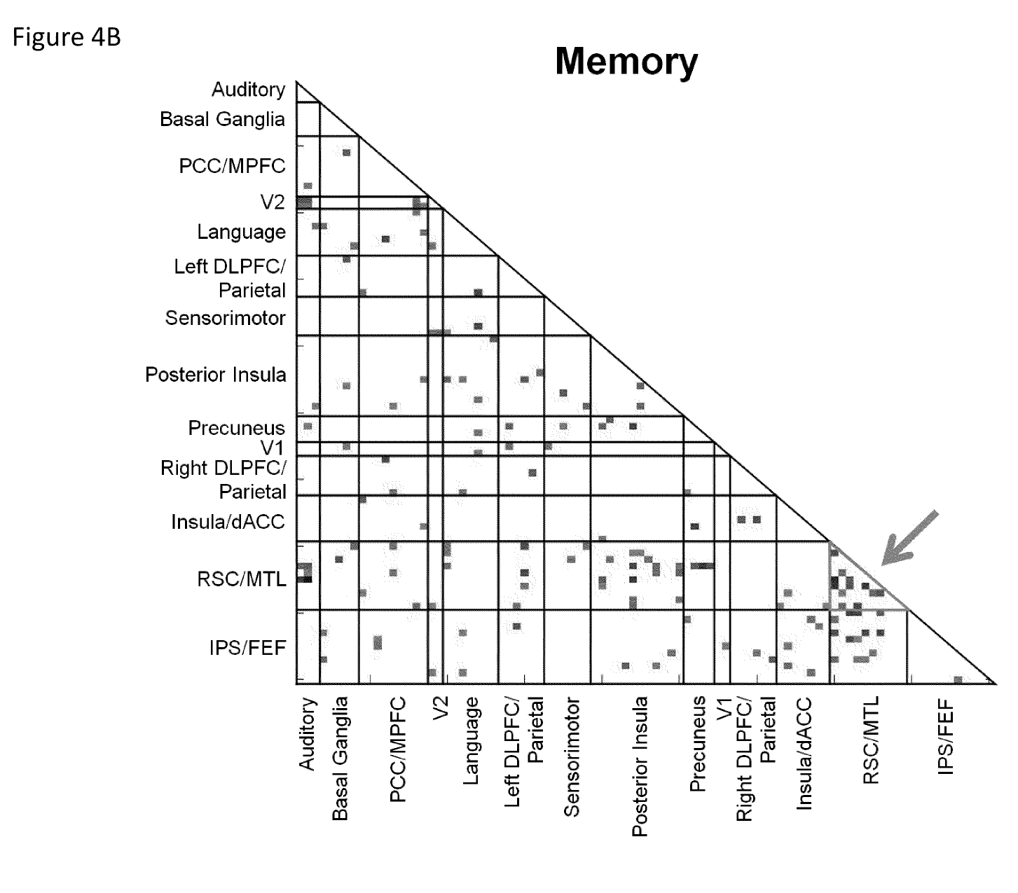

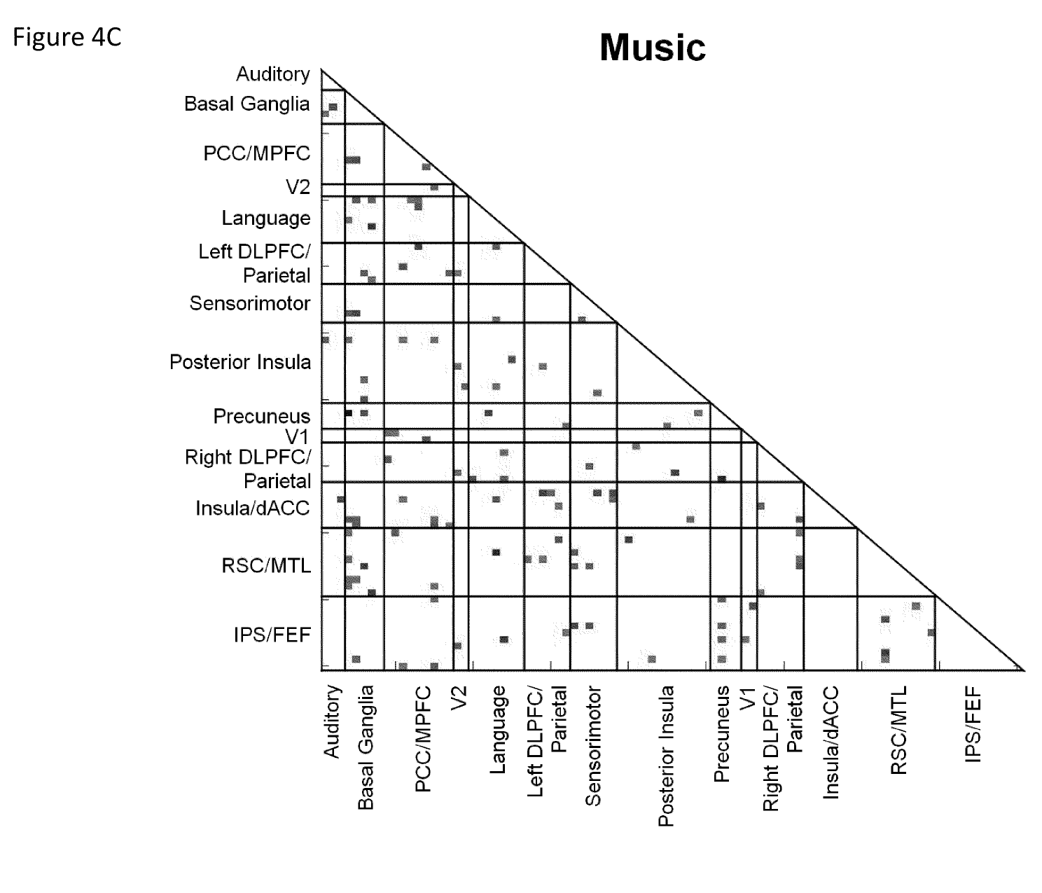

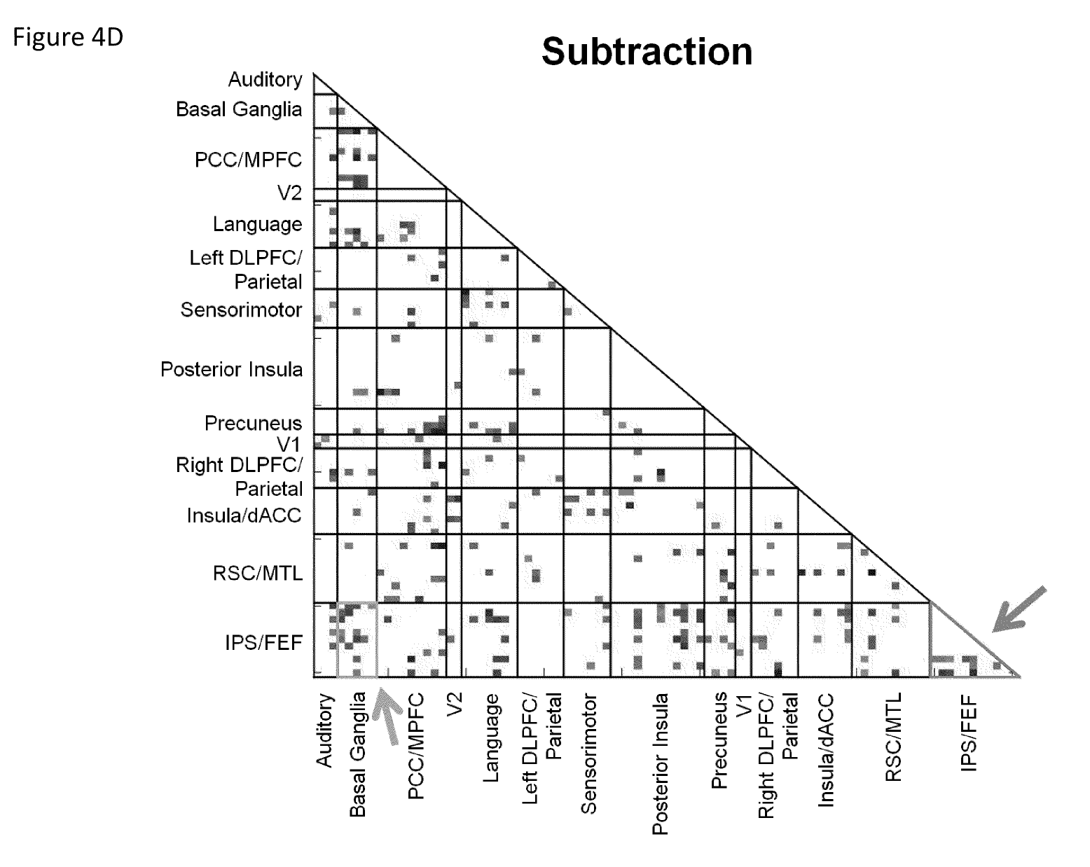

Group-level state matrices. We created our classification algorithm by selecting cells of interest for each of the four cognitive states studied in the first cohort of subjects. The classifier was not trained on the spatial navigation task. For each cognitive state we performed a one-sample t-test across all subjects for each of the 3960 cells and retained cells that were significant at an FDR-corrected p-value of 0.01. Any cells that were significant for more than one cognitive state were excluded. This resulted in state-specific cells with strong positive or negative correlations that were consistent across subjects and unique to a particular cognitive state. These criteria identified 187 cells of interest for rest, 147 cells of interest for memory, 114 cells of interest for music, and 265 cells of interest for subtraction (see FIG. 4). The classifier parameters were developed on the full 14-subject training dataset and then validated in both an LOOCV analysis and on the independent cohort.

Classifier Validation

Classification of four subject-driven cognitive states. An individual's four cognitive states were classified by deriving an overall measure of their functional connectivity (FC) within each of the four group-level state matrices. This was tested with two different cohorts of participants to confirm the generalizability of the classification algorithm used: the original cohort of 14 subjects using leave-one-out cross-validation (LOOCV) and the independent validation cohort of 10 subjects. On a subject-by-subject basis, each of an individual's four scan matrices was assigned to the group-level state matrix that it best matched based on a spatial correlation fit score. To calculate the fit of a given individual scan matrix to a specific group-level state matrix, we first examined FC within the cells of interest for the group-level state matrix and determined whether the sign of the individual cell FC agreed with the sign of the group-level cell FC.

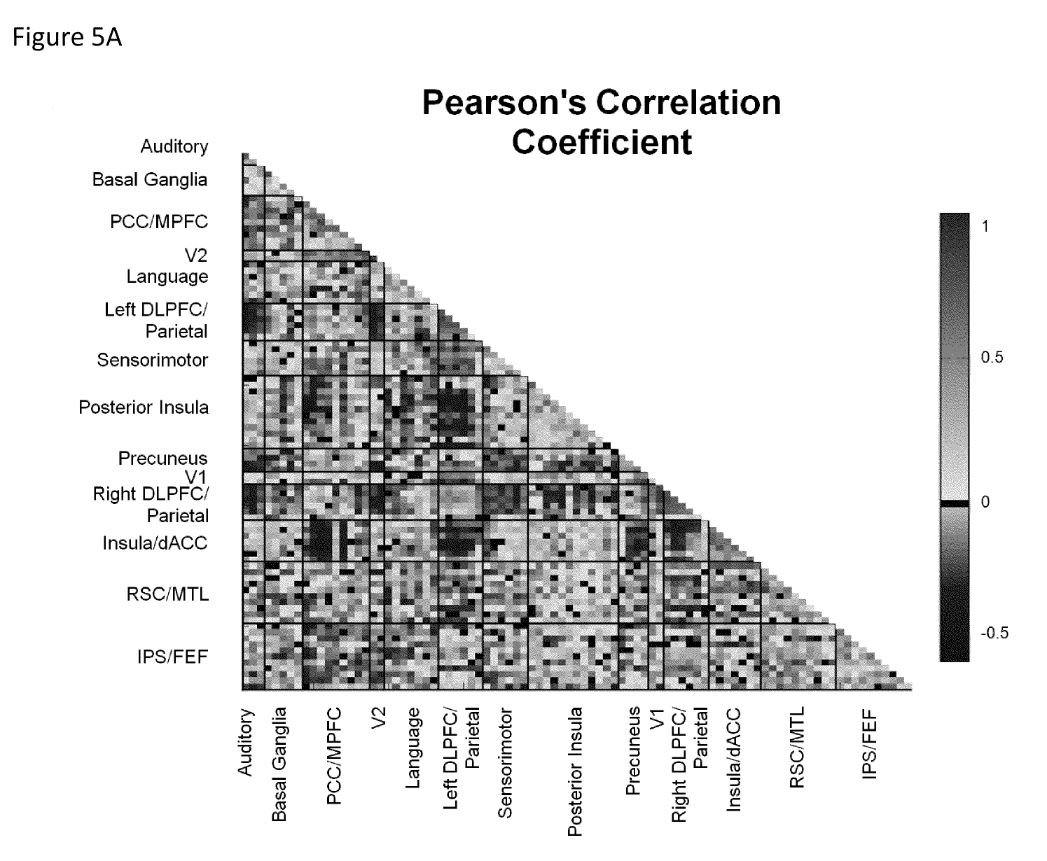

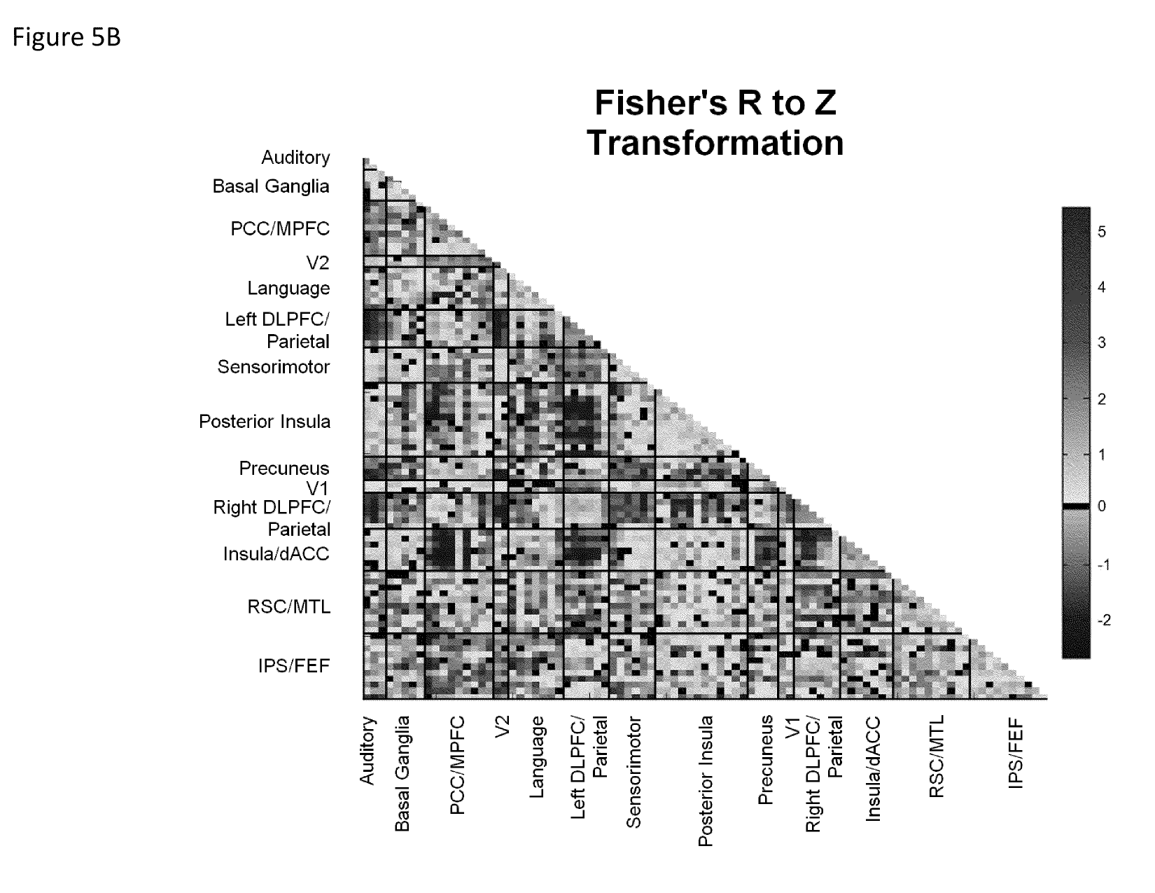

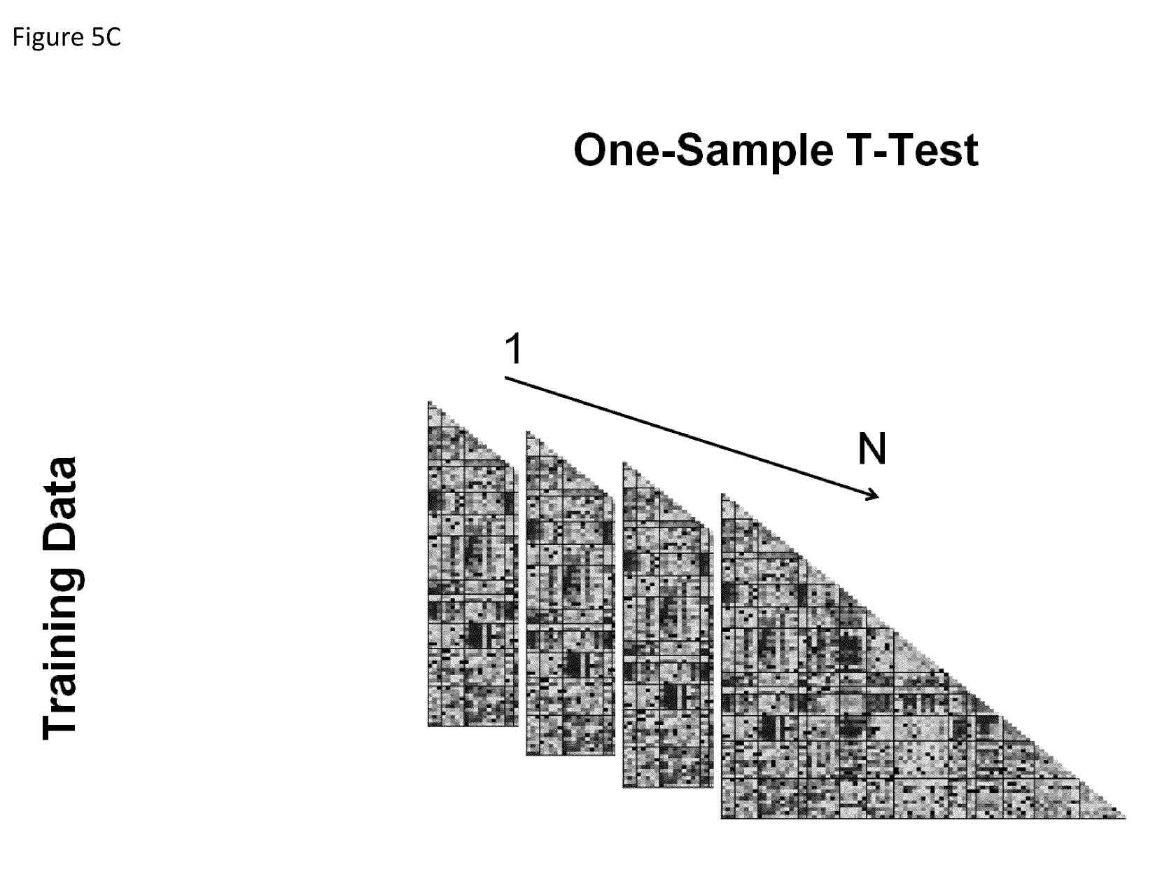

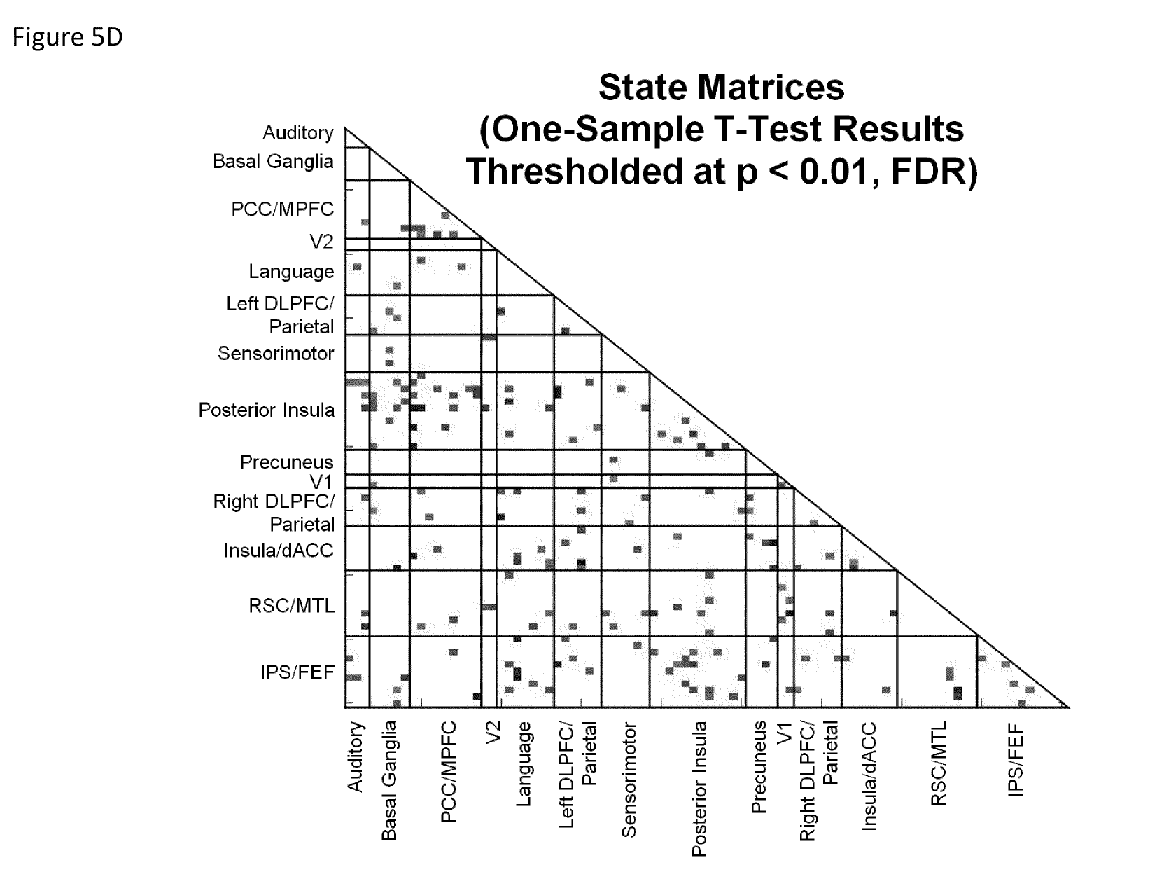

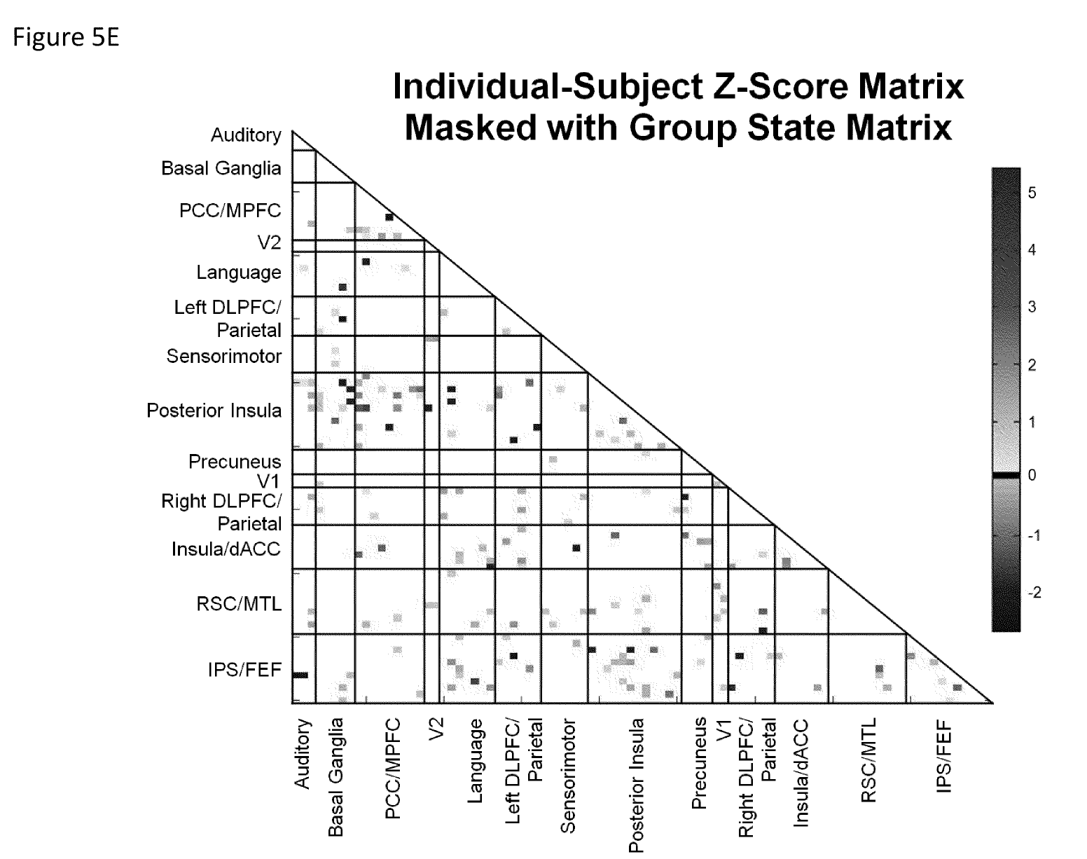

Cells whose FC sign agreed with the group-level matrix's cell sign were identified as “correct” and cells whose sign did not agree as “incorrect”. To derive the fit score, each cell was multiplied within the individual state matrix by the z-score in the corresponding cell of the group-level state matrix. This allowed us to weight each cell in the individual state matrix by the FC strength predicted by the group-level state matrix. We then took the sum of the absolute values for all correct cells multiplied by the proportion of correct cells, and subtracted the sum of the absolute values of all incorrect cells multiplied by the proportion of incorrect cells. Because the algorithm calculates the fit score from the average connectivity in the cells of interest, the algorithm is unbiased by the number of cells in each group matrix. For each subject, the scan that had the highest fit score for a group-level state matrix was assigned that cognitive state. A binomial test was used to determine the significance of the classification accuracy. A flow chart of the classification algorithm is provided in FIG. 5.

For the LOOCV the 4 group-level state matrices were calculated 14 different times such that a given subject's scans were compared to group-level state matrices generated from the other 13 subjects. Although LOOCV is a standard method for demonstrating classification generalizability (Mitchell et al., 2003; Mourao-Miranda et al., 2005), it is prone to cohort effects as the classifier may be over-fit to a dataset that is not fully representative of the broader population (Davatzikos et al., 2005; Hastie et al., 2009). Accordingly, we also applied this classification algorithm to a completely independent cohort of 10 new subjects acquired several months after the original cohort described above. For the validation cohort classification we used the group-level state matrices shown in FIG. 4 derived from all 14 subjects in the initial cohort.

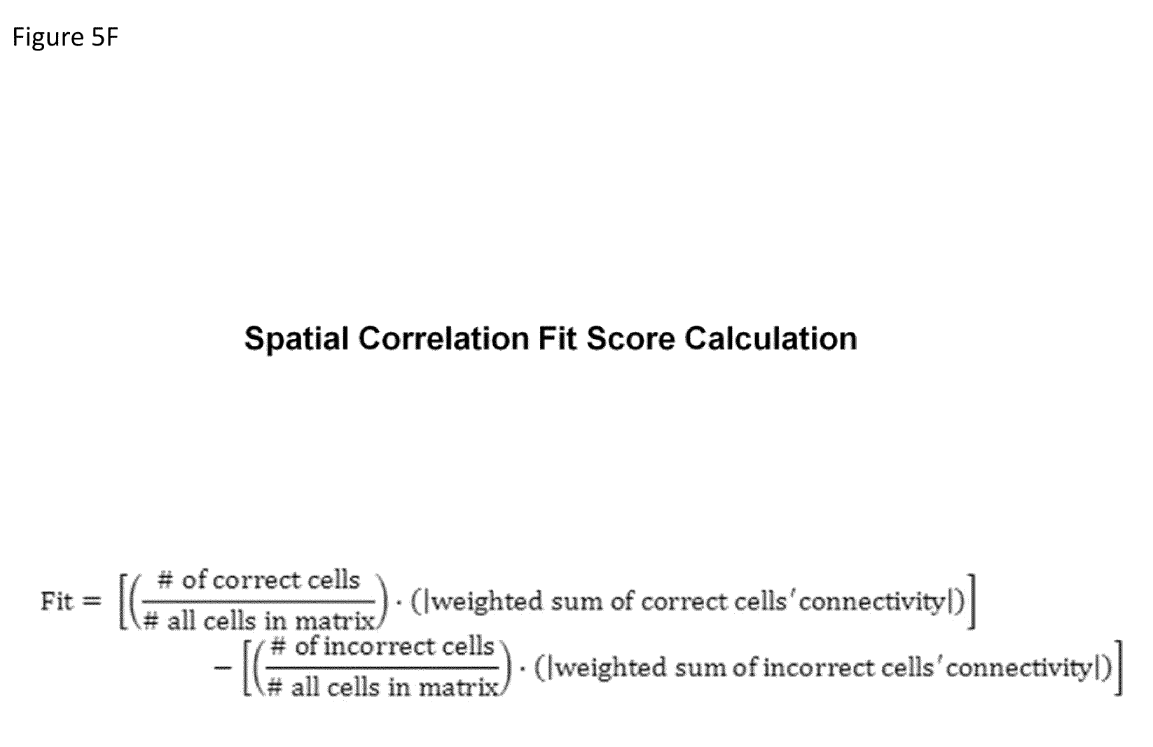

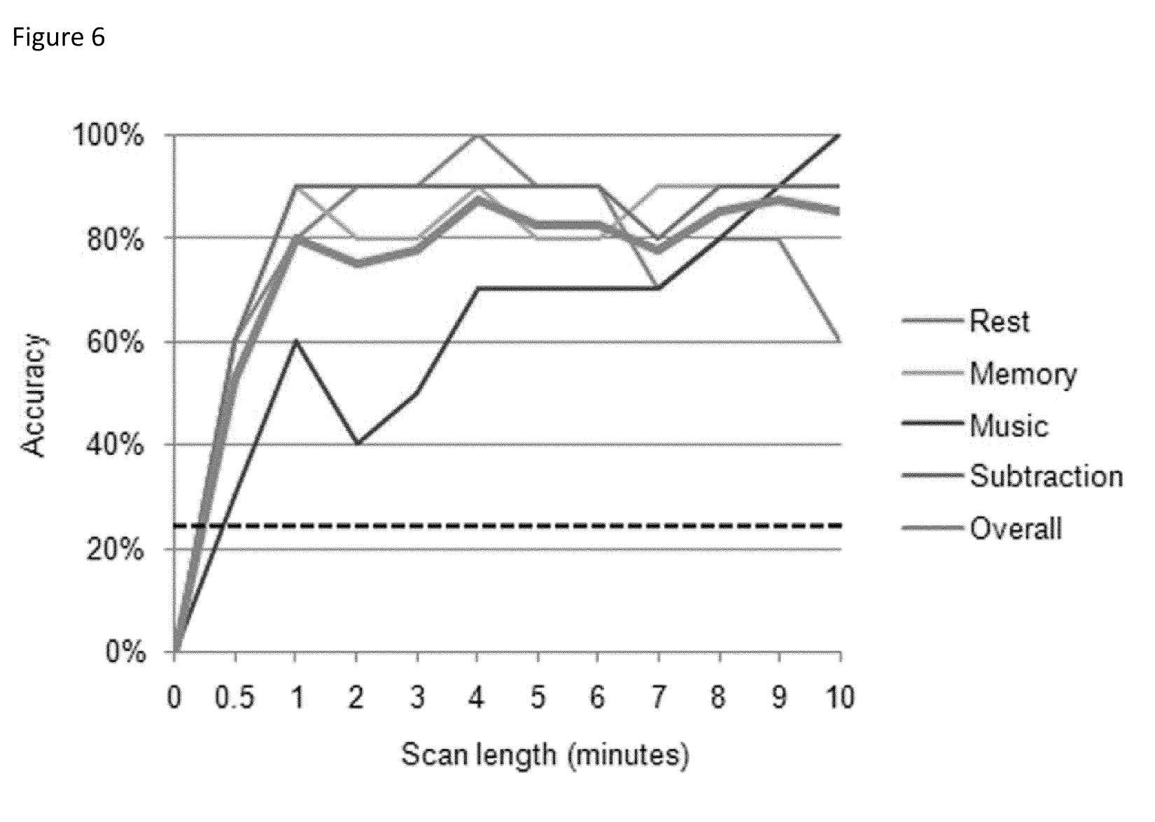

Classification accuracy as a function of scan length. To determine the influence of scan duration on classification accuracy, we repeated classification of the validation cohort at 11 increasingly shorter scan durations ranging from 10 minutes down to the first 30 seconds (FIG. 6).

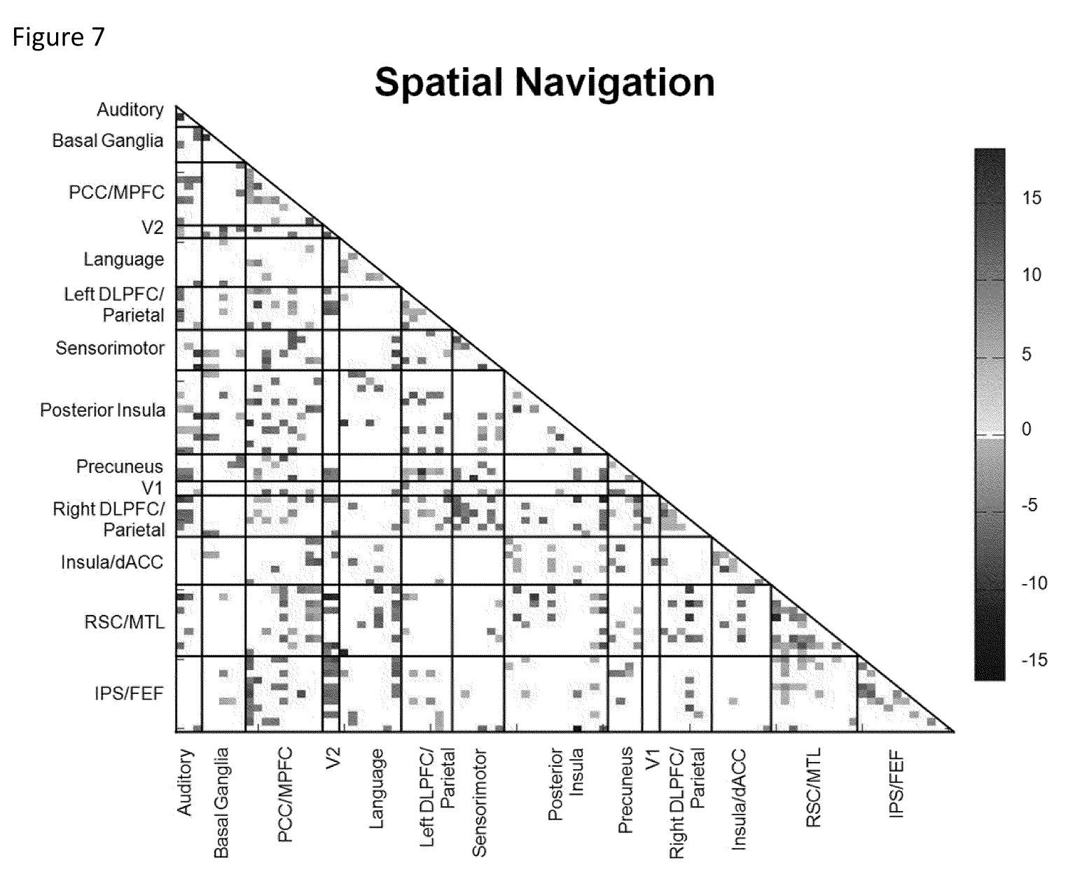

Rejecting a novel, fifth cognitive state. The 10 subjects from the validation cohort also completed a self-driven spatial navigation task in which they were asked to imagine walking through the rooms of their home. This task was used to assess whether the classifier was sufficiently specific to exclude or reject a novel cognitive state from the four states on which it was trained. We calculated an individual-subject spatial navigation matrix for each of the 10 subjects, and included this matrix with the 4 other scan matrices in a best-fit analysis. On a subject-by-subject basis, each of the 5 individual scans was assigned a fit score to each of the 4 group-level state matrices. In this classification analysis, given that there were only 4 group-level state matrices and 5 scans, we forced unique assignments of the individual scans to the group-level state matrices using a “winner-take-all” approach. If two of an individual's scans matched to the same group-level matrix, the better match was selected and the second scan was assigned to its second-best match. The individual scan that did not fit any of the group-level state matrices better than the other individual scans was classified as the spatial navigation scan. Note that this “winner-take-all” algorithm is less stringent than the “best-fit” algorithm used for our main classification analyses and described above under “Classification of four subject-driven cognitive states”. A one-sample t-test for the spatial navigation scan matrix is provided in FIG. 7.

Classification with structural ROIs. The “best-fit” algorithm described above was implemented to create group-level state matrices for the original cohort and classify the four cognitive states in the validation cohort using 112 structurally-defined ROIs from the AAL Atlas (Tzourio-Mazoyer et al., 2002).We used a binomial test to determine the significance of the classification accuracy, and performed a paired-samples t-test to compare accuracy when using structural ROIs with accuracy when using functional ROIs.

Group-level contrasts of rest and memory states. Connectivity between and within ICNs was compared using a paired-sample t-test for the memory state and the rest state of the 14 subjects used to train the classifier (FIGS. 3D and 3E). To compare connectivity between all 90 ROIs in the rest and memory states (FIG. 3D), we performed a paired-sample t-test between the states for each of the 3960 pairwise correlation cells. Significant cells were determined by using an FDR-corrected p-value of 0.05. To compare connectivity within the RSC/MTL ICN (FIG. 3E), we performed ICA on each subject's rest and memory states. We fixed the number of independent components at 30 for each subject. We then used an automated template-matching procedure to select the RSC/MTL ICN for each scan (Greicius et al., 2004) using the group-level RSC/MTL as a template, and compared the connectivity within this ICN for the subjects' rest and memory scans by performing a paired-sample t-test in SPM5. This analysis was masked to a one-sample t-test of the network derived from both states so that results would only reflect changes within the RSC/MTL network. Significant clusters of connectivity within the group-statistical map were determined by using the joint expected probability distribution (Poline et al., 1997) with height (p<0.01) and extent (p<0.01) thresholds, corrected at the whole-brain level.

Between-Group Classification to distinguish subjects suffering from Alzheimer's disease from control subjects. Similar methods were used to generate the between-group classifier used to distinguish subjects suffering from Alzheimer's disease from neurotypical control subjects except that in addition to using a group-level state matrix for each group we also generated a mean difference matrix showing cells whose correlations differed between the two groups. Classification can be performed by determining which single group-level matrix a subject's matrix best matches (as in the cognitive state classification described above). Alternatively, classification can be performed by generating a range of scores for controls and subjects suffering from Alzheimer's disease based on their fit to the group difference matrix. A single subject's fit to the difference matrix can then be assessed to determine if it falls in the control range or in the Alzheimer's disease range.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, temperature is in degrees Centigrade, and pressure is at or near atmospheric.

### Example 1: Parcellation of Gray Matter Into Functional Regions of Interest (ROIs) to Create a Whole-Brain Functional Connectivity Matrix

Resting-state fMRI analyses have revealed a large set of distinct brain networks that correspond to several critical brain functions including vision, hearing, language, emotion, and memory (Beckmann et al., 2005; Damoiseaux et al., 2006; Greicius et al., 2003; Seeley et al., 2007). We have identified 14 such networks consistently in our data and then thresholded each network to generate between 2-12 ROIs per network, When the ROIs within each network were combined and mapped across the brain (see FIG. 1), we were able to cover the vast majority of cortical and sub-cortical gray matter.

The spatial resolution of this approach will likely continue to improve with advances in fMRI acquisition and analysis. Many of the ROIs used here are still relatively large (FIGS. 1 and 2) and can likely be subdivided further with increasingly sophisticated parcellation approaches. Combining resting-state fMRI with diffusion tensor tractography (Greicius et al., 2009; Rushworth et al., 2006) and self-clustering functional connectivity algorithms (Cohen et al., 2008) represent two promising approaches to more finely parcellating gray matter into increasingly indivisible mesoscopic functional units. A third approach would entail mandating a higher model order in the group ICA, so that instead of identifying 14 networks from 30 components as was done here, one might, for example, identify 20 networks in 50 components (Kiviniemi et al., 2009; Smith et al., 2009). With the whole-brain connectivity matrix approach defined here, doubling the number of ROIs from 90 to 180 would increase the matrix size exponentially from 3960 cells to 16,020 cells which may further enhance discriminability between cognitive states.

### Example 2: Classification of Brain States

Subject-driven tasks drive connectivity changes. The whole-brain functional connectivity approach, as described in Example 1, does not require controlling when subjects perceive or respond to a stimulus; it can, therefore, be applied to cognitive states that are free-streaming and subject-driven. This approach was successfully applied to classify four different subject-driven cognitive states based on their pattern of whole-brain connectivity. Subjects were scanned under the following four subject-driven conditions: undirected rest, retrieval of recent episodic memories, serial subtractions, and (silent) singing of music lyrics. The imaging data were acquired in continuous ten-minute runs with no stimulus presentation and no investigator-imposed temporal landmarks other than the start and end of the scan. Patterns of within- and between-ICN connectivity were used to train a classification algorithm on data from 14 subjects.

For each of the 4 scans in each of the 14 subjects a 90×90 matrix of pairwise ROI correlations was calculated (FIGS. 3A and 3B). These matrices can be compared directly within a subject to reveal changes in connectivity strength between two subject-driven cognitive states, as highlighted for the rest and memory states in FIG. 3C. Group-level analyses confirmed these findings, revealing significant connectivity differences across the 90×90 matrix both within and between ICNs (FIG. 3D, p<0.05, FDR corrected). In FIG. 3E, a specific intrinsic connectivity network (ICN) was highlighted that included ROIs in the retrosplenial cortex (RSC) and medial temporal lobe (MTL) and that showed significantly increased connectivity in the memory state compared to rest (p<0.01, corrected). There were no clusters in this ICN that showed significantly increased connectivity in the opposite contrast (rest>memory).

Group-level state matrices. FIG. 4 demonstrates group-level connectivity matrix patterns that were consistent across 9 of 14 subjects and unique to each of the four cognitive states. These four matrices were then used to determine which of the four cognitive states 10 new subjects were engaged in based on how well a given subject's connectivity matrix matched one of the four group-level state matrices shown in FIG. 4. In the group-level memory state matrix (FIG. 4B, orange arrow) several cells corresponding to correlations within the RSC/MTL ICN survived as would be expected from the group-level increases in this ICN during the memory state compared to rest (FIGS. 2D and 2E). Equally important to the cells showing state-specific within-ICN correlations are the numerous cells showing state-specific between-ICN correlations. In FIG. 4D (blue arrow) we emphasize increased connectivity during the subtraction task within an ICN that includes intraparietal sulcus (IPS) and prefrontal regions. In addition to increased intra-network connectivity, the subtraction task elicited increased connectivity between the IPS/prefrontal ICN and the basal ganglia ICN (FIG. 4D, green arrow).

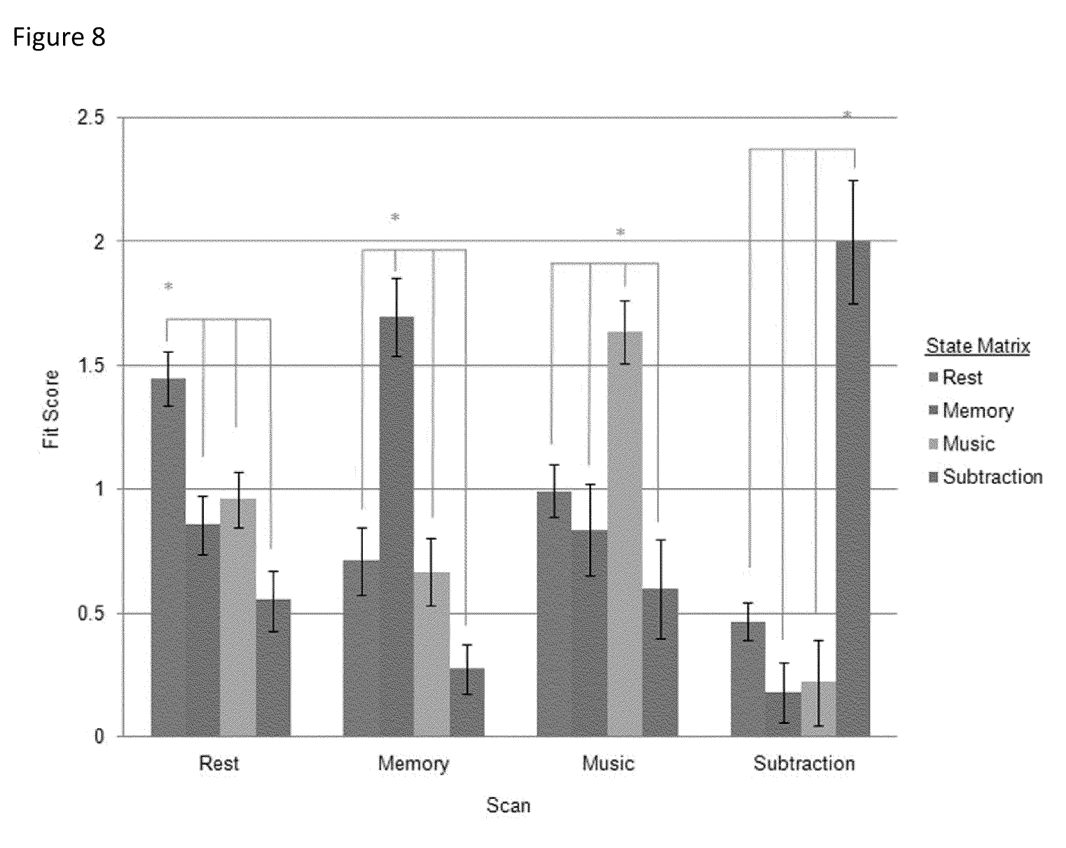

Classification of four subject-driven cognitive states. Using the pattern-recognition classifier approach, 84% of the states were correctly identified in the LOOCV analysis (47 of 56 states; p<0.001). Additionally, we used the group-level state matrices generated from the original cohort, shown in FIG. 4, to classify the four cognitive states in an independent cohort of 10 new subjects acquired several months after the original cohort. In the independent cohort, 85% of the states were correctly classified (34 of 40 scans; p<0.001). The mean state matrix fit scores for each of the 4 scan types across the 10 subjects are shown in FIG. 8.

Classification accuracy as a function of scan length. With a goal of applying this approach to more naturalistic (briefer) subject-driven cognitive states we next examined the classifier accuracy over shorter scan durations in the independent cohort. Classification accuracy remained as high as 80% using only the first minute of data. Classification accuracy by scan length is shown in FIG. 6, indicating that a high level of accuracy can be obtained with scan lengths as short as 30 seconds.

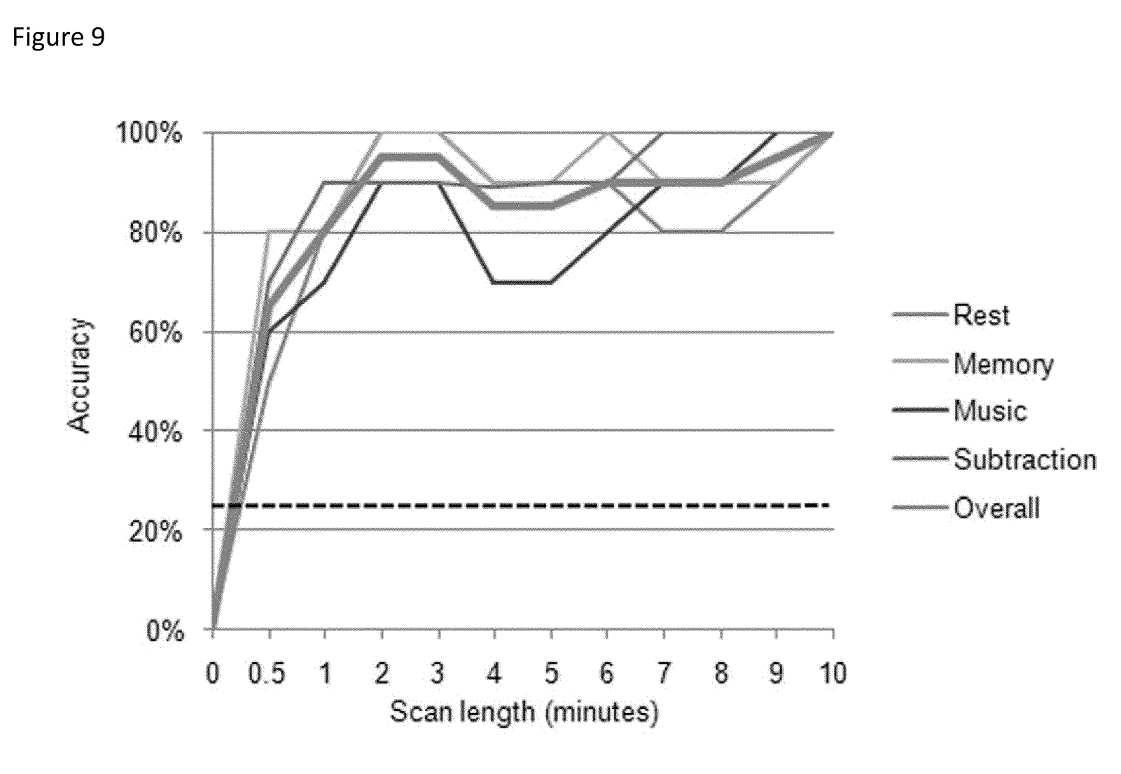

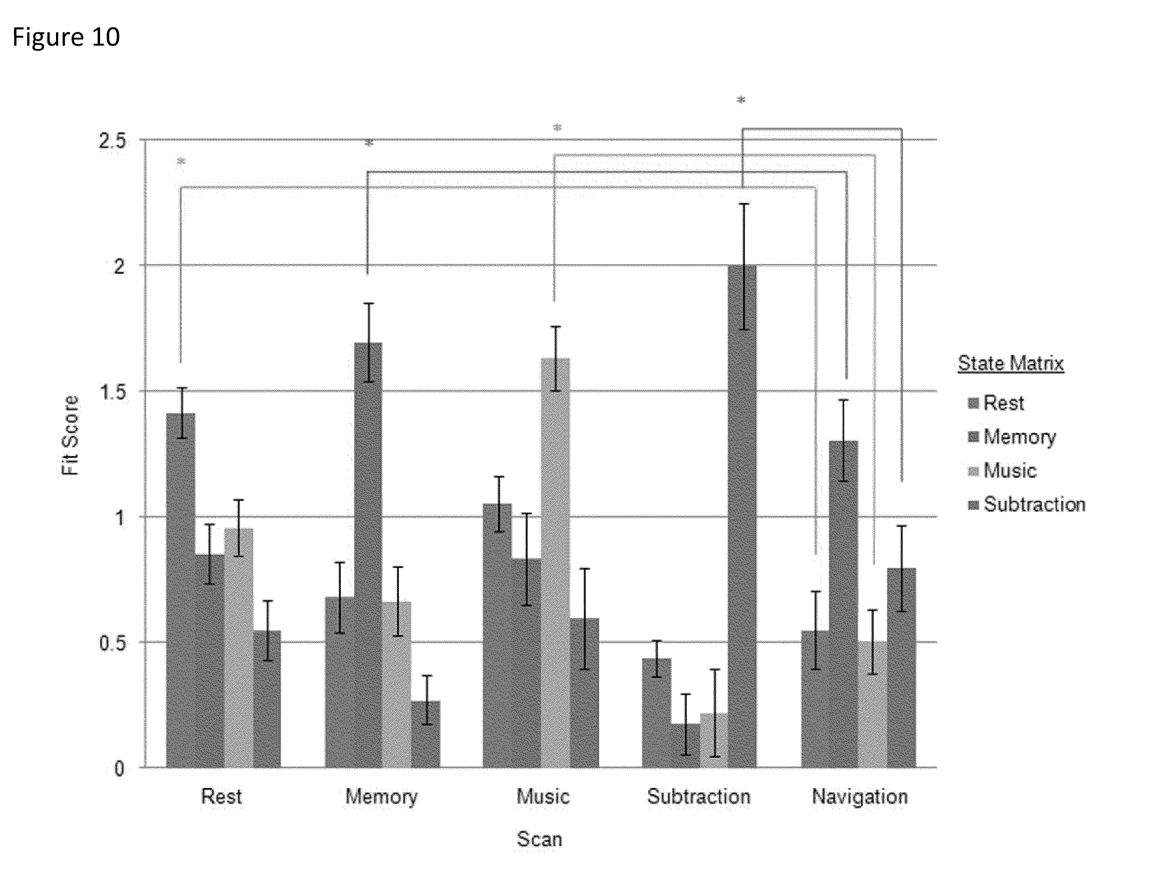

Rejecting a novel, fifth cognitive state. In an embodiment of the invention, where spatial navigation scans were added, 46 of 50 scans in the validation cohort were correctly classified yielding a classification accuracy of 92% (p<0.001). Note that classification accuracy is higher here than in our main 4-way classification because we used a winner-take-all approach. When applied to the 4-way classification, the winner-take-all approach results in 100% accuracy for both the LOOCV and independent cohort classification analyses (FIG. 9).The mean state matrix fit scores for each of the 4 scan types were significantly greater than the novel cognitive state (FIG. 10). For one participant, the spatial navigation task was confounded with the memory task; for another, the spatial navigation task was confounded with the subtraction task. The group-level state matrix for the spatial navigation task was not used to train the classifier, but is shown in FIG. 5.

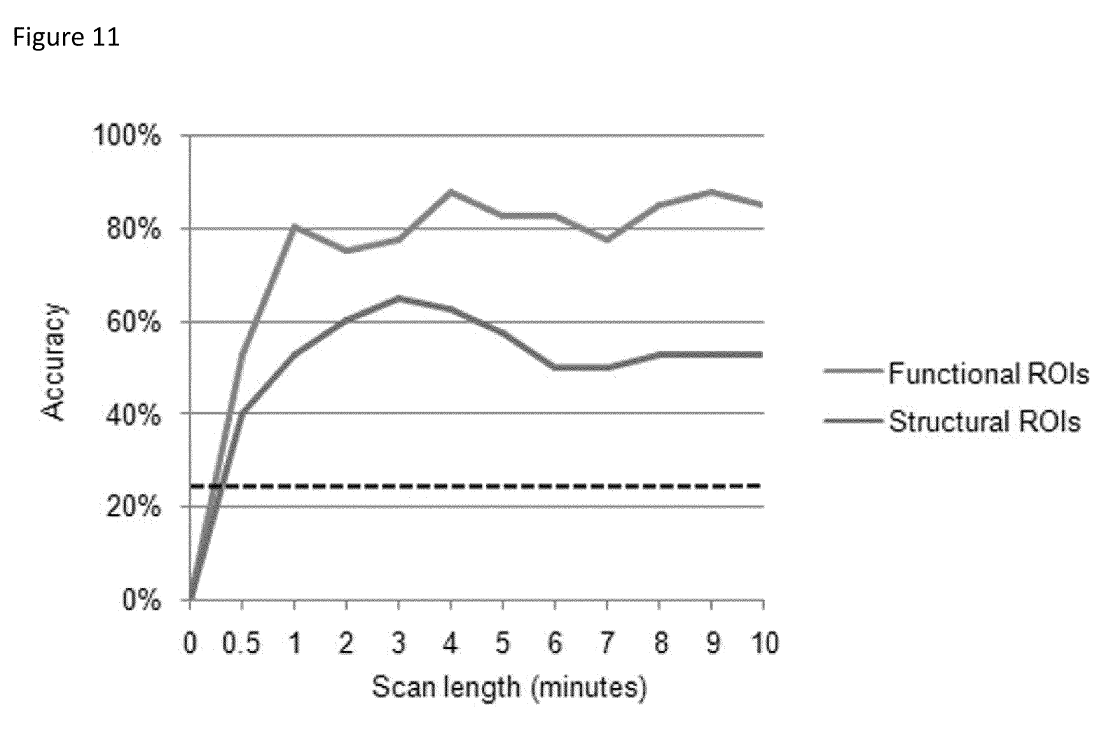

Comparison of functional and structural ROIs in classification. Classification accuracy with the structural ROIs reached significance for all scan lengths (p<0.001); however, the highest classification accuracy achieved with the structural ROIs was 65% (26 of 40 states correctly classified, FIG. 11). Additionally, a paired-samples t-test revealed that classification with the structural ROIs was significantly less accurate than classification with the functional ROIs (p<0.001).

### Example 3: Classification of Neuropsychiatric Conditions and Their Progression or Response to Treatment

The same approach used to classify different cognitive states can be used to classify subjects, who suffer from or might be at risk of developing neuropsychiatric diseases or disorders and controls based on their whole-brain connectivity matrix.

Neurodegenerative diseases such as Alzheimer's disease are examples of neuropsychiatric diseases, while chronic pain exemplifies a neuropsychiatric disorder. The determination of specific cognitive traits in neurotypical subjects, who represent healthy control subjects with a neurotypical profile, in comparison to specific cognitive traits in neuro-atypical subjects, who deviate from a neurotypical profile in some form, can provide important guidance in the clinical diagnosis of neuropsychiatric diseases and disorders, in the monitoring of neuropsychiatric disease progression and in the monitoring of neuropsychiatric treatment success.

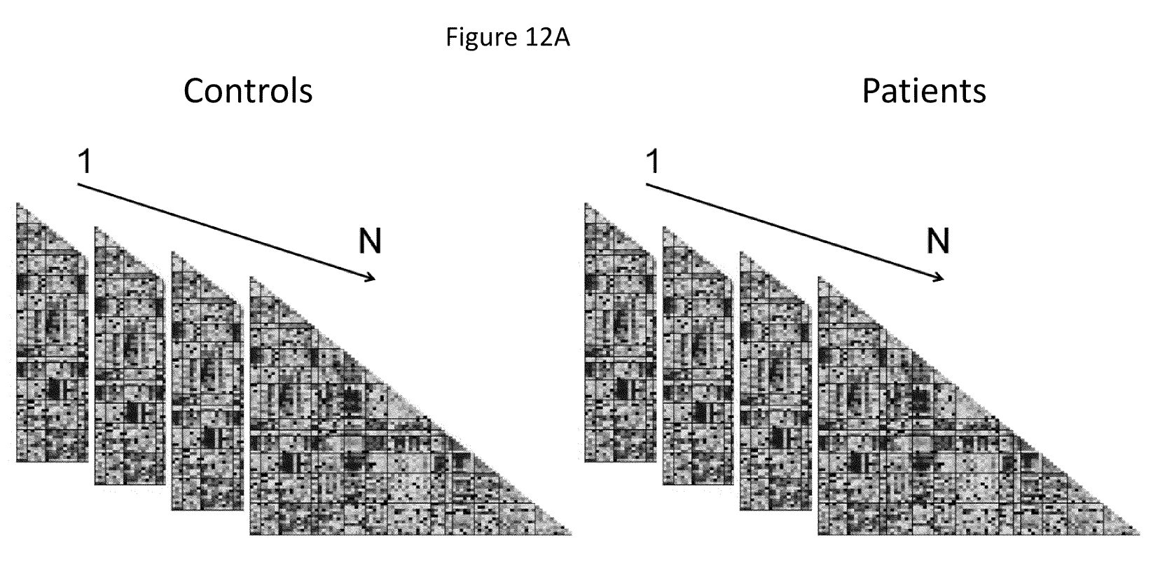

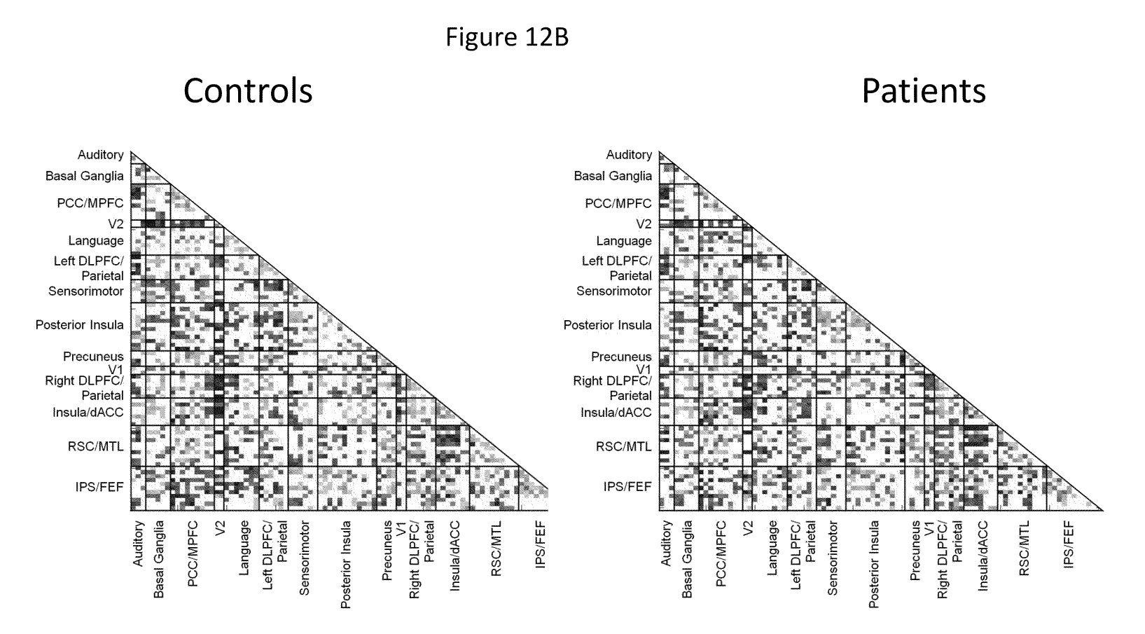

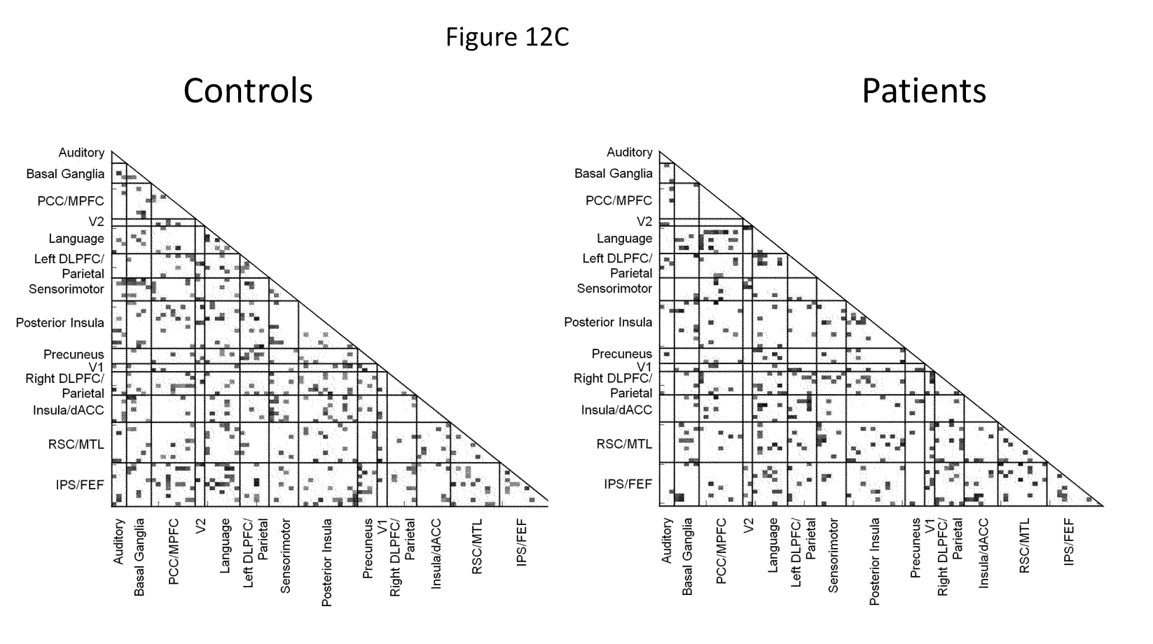

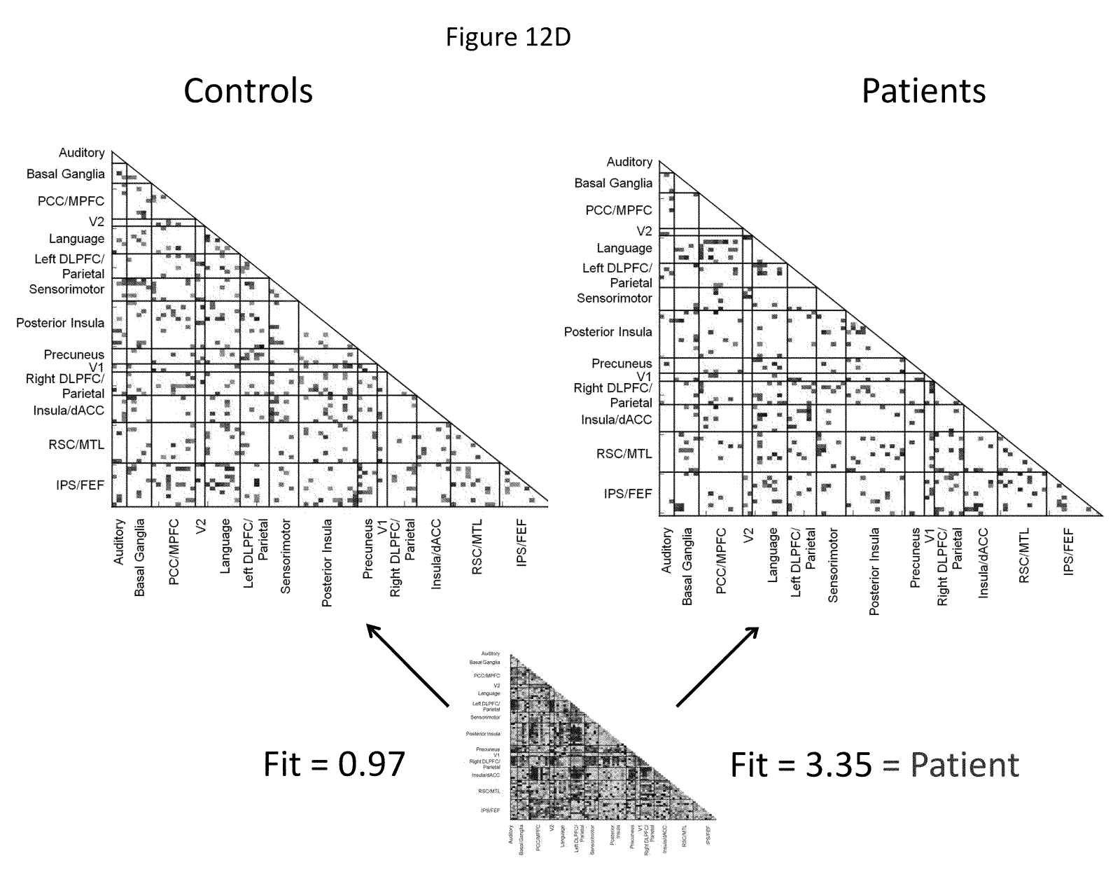

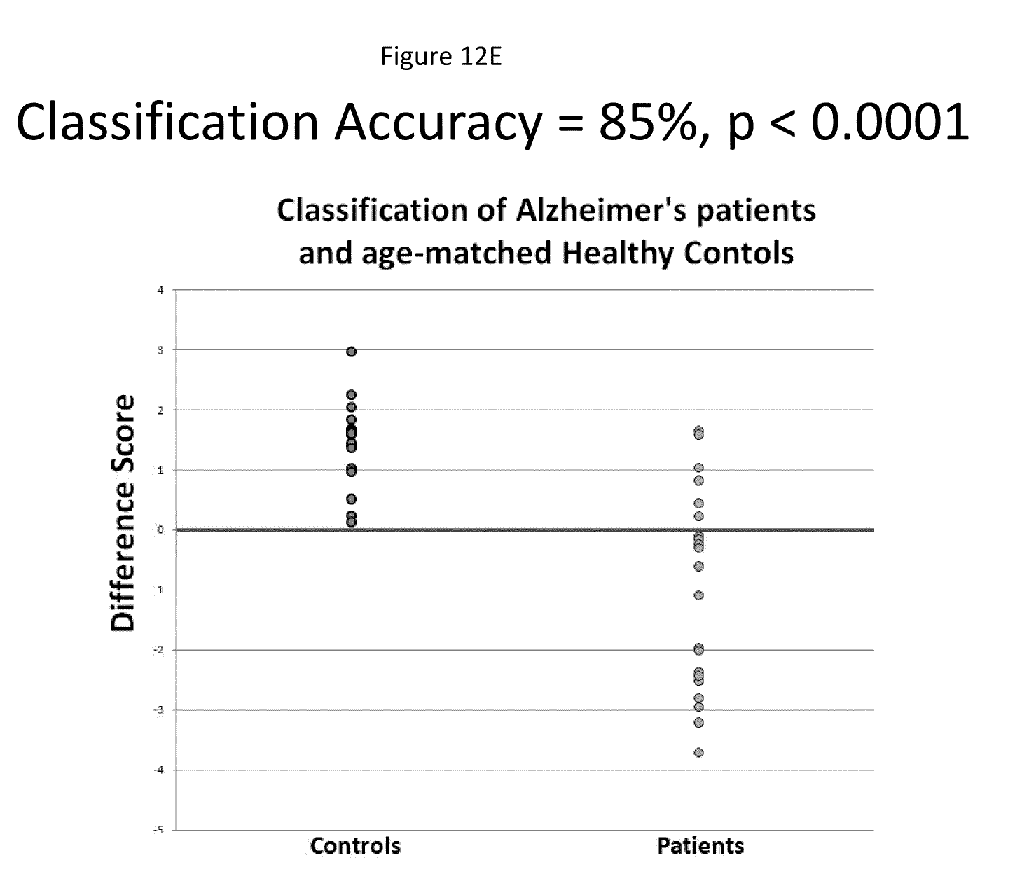

Distinguishing subjects who suffer from Alzheimer's disease from healthy, neurotypical controls. Using resting-state data, group-level state matrices were developed for healthy, neurotypical controls and for subjects suffering from Alzheimer's disease. Classification with whole-brain functional connectivity was 85% accurate in distinguishing subjects suffering from Alzheimer's disease from healthy, neurotypical controls (FIG. 12).

Using a similar approach to that outlined in FIG. 5, whole-brain resting-state connectivity matrices were defined for a group of subjects suffering from Alzheimer's disease and a group of healthy, neurotypical, older control subjects using one-sample t-tests (FIG. 12A). These group-level connectivity matrices were thresholded (FIG. 12B) and cells that appeared in both matrices were removed (FIG. 12C). A single-subject's whole-brain resting-state functional connectivity matrix was then compared to each of the group-level matrices allowing us to calculate a fit score for each subject (FIG. 12D). A given subject was classified as a control, if his fit score to the control matrix was greater than his fit score to the Alzheimer's matrix (difference score>0). If a subject's difference score was less than zero (better fit to the Alzheimer's matrix),then he was classified as a subject suffering from Alzheimer's disease. Using this approach, 85% of subjects were correctly classified (FIG. 12E).

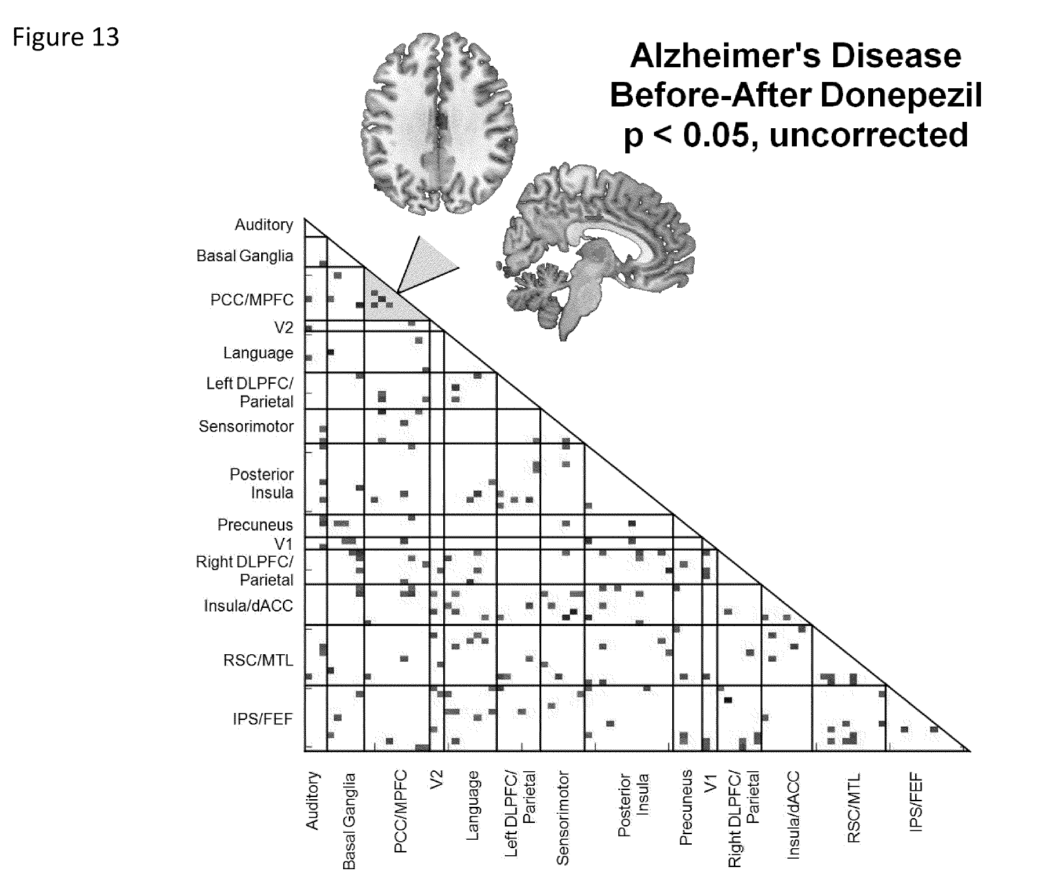

In further studies, subjects who suffer from Alzheimer's disease underwent resting state fMRI before and 6 weeks after treatment with donepezil (Aricept®), a centrally acting reversible acetylcholinesterase inhibitor used for the palliative treatment of mild to moderate Alzheimer's disease. FIG. 13 shows a paired-sample t-test of the whole-brain connectivity matrix of those subjects identifying regions that had significantly increased (blue cells) or decreased (red cells) connectivity following treatment with donepezil. The grey triangle highlights regions in a brain network targeted by Alzheimer's disease whose connectivity increased after treatment.

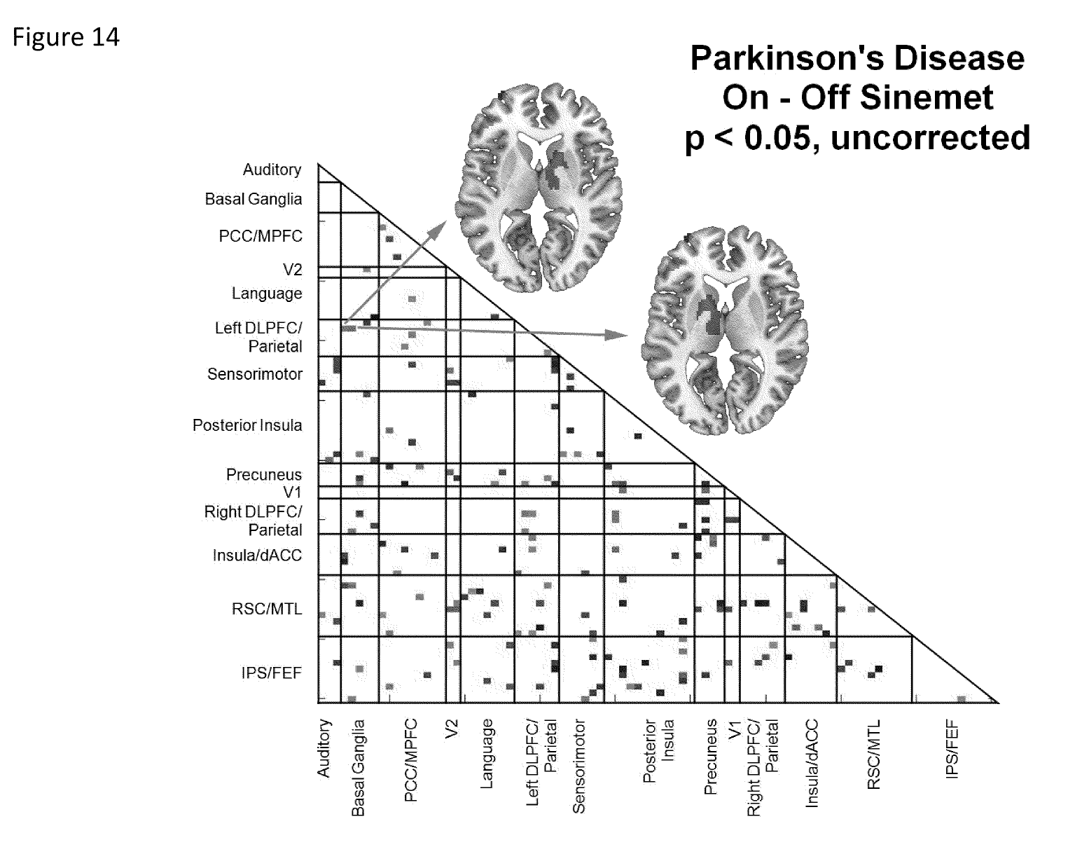

Detecting response in subjects suffering from Parkinson's disease to anti-Parkinson's disease treatment using whole-brain functional connectivity analysis. Subjects were scanned during treatment with Sinemet® and off treatment with Sinemet®, a carbidopa/levadopa combination to treat Parkinson's disease. FIG. 14 shows a paired-sample t-test of the whole-brain connectivity matrix identifying regions that had significantly increased (red cells) or decreased (blue cells) connectivity following treatment with sinemet. The green arrows highlight cells which reflect increased connectivity between the bilateral basal ganglia and the prefrontal cortex when the subjects received Sinemet® treatment.

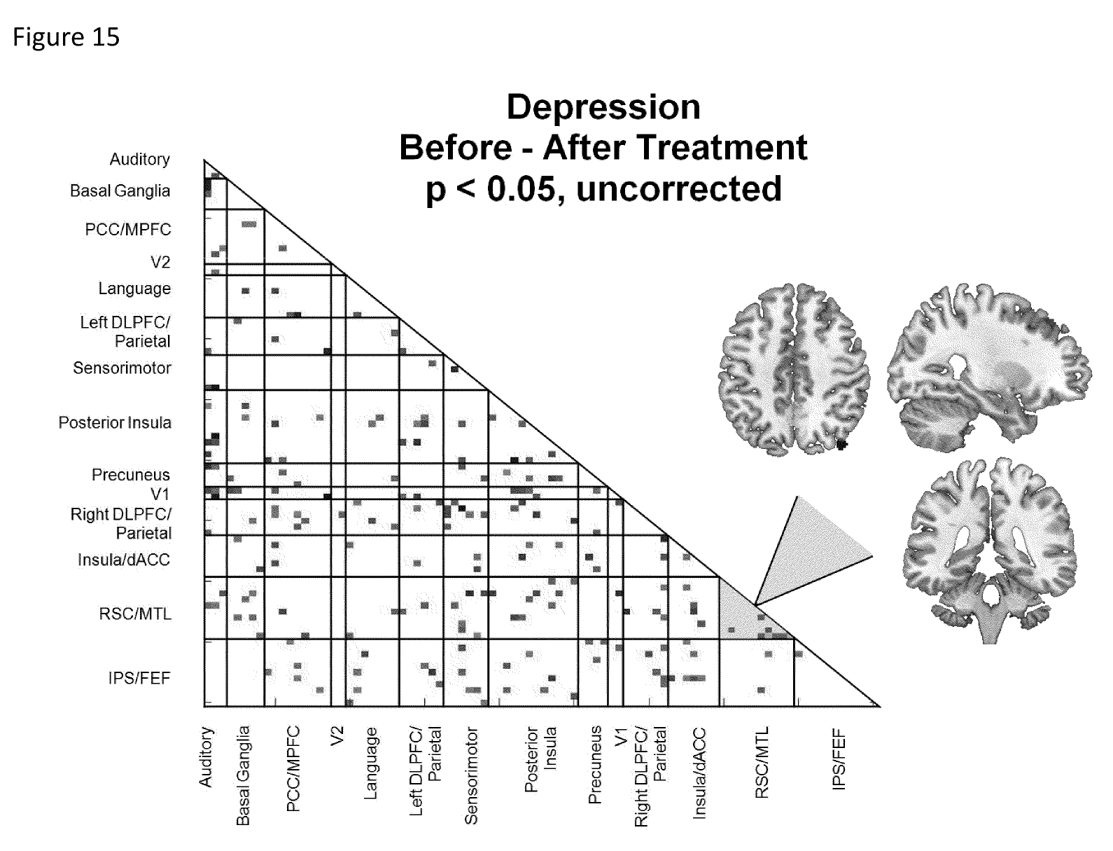

Detecting response in subjects suffering from depression to antidepressant treatment using whole-brain functional connectivity analysis. FIG. 15 shows a paired-sample t-test of the whole-brain connectivity matrix identifying regions that had significantly increased (blue cells) or decreased (red cells) connectivity in subjects suffering from depression following treatment, in comparison to before treatment, with the antidepressant citalopram (Celexa®), a selective serotonin reuptake inhibitor. The grey triangle highlights regions in a medial temporal lobe memory network whose connectivity increased after treatment.

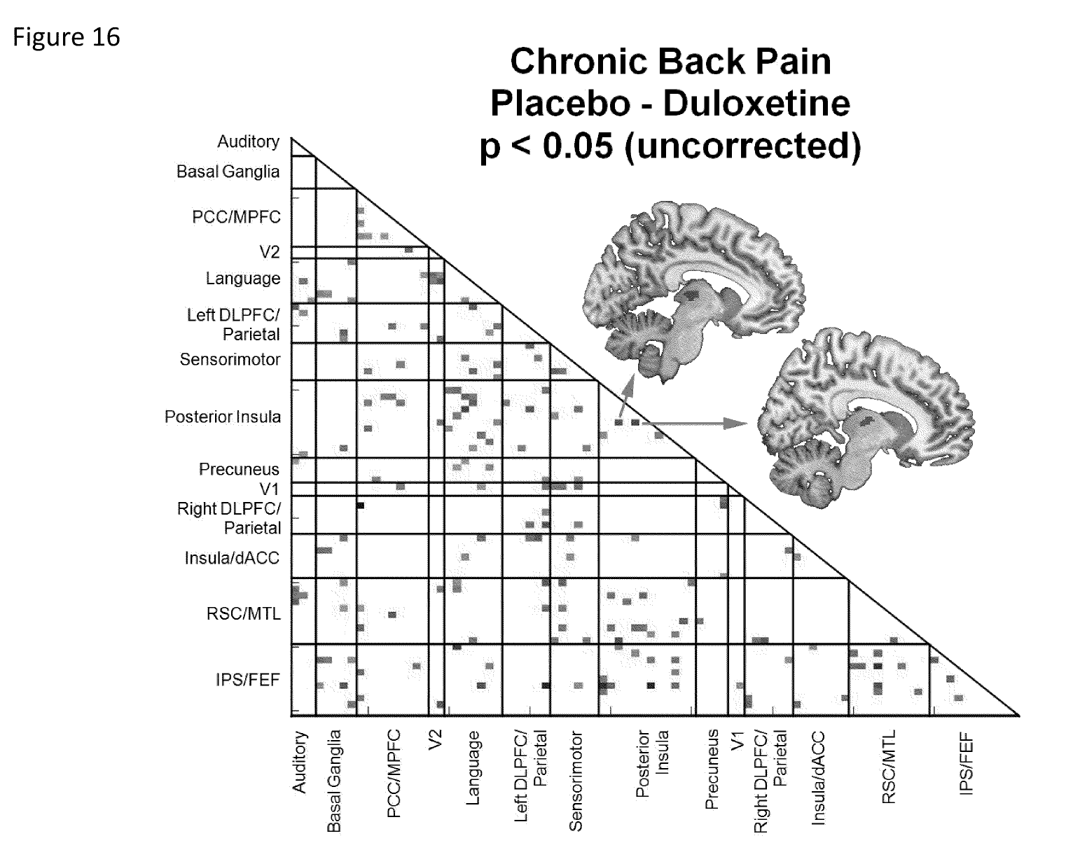

Detecting response in subjects suffering from chronic pain to pain relieving agent using whole-brain functional connectivity analysis. FIG. 16 shows a paired-sample t-test of the whole-brain connectivity matrix identifying regions that had significantly increased (blue cells) or decreased (red cells) connectivity in subjects suffering from back pain following treatment with duloxetine compared to placebo. Duloxetine (Cymbalta®) is a non-narcotic, non-NSAID pain relieving agent that is indicated, among other indications, for chronic musculo-skeletal pain. The green arrows identify cells that reflect increased connectivity between bilateral sensory regions and the thalamus in subjects when treated with duloxetine compared to when treated with placebo.

Distinguishing subjects who suffer from chronic pain from healthy, pain-free control subjects. Using resting-state data, group-level state matrices were developed for healthy, pain-free control subjects and for subjects suffering from chronic pain. Chronic pain includes lower back pain, migraine,fibromyalgia, arthritis pain, malignant pain, neuropathic pain and similar conditions. The classification of subjects using whole-brain functional connectivity was 65% accurate in distinguishing subjects suffering from chronic pain from control subjects.

By acquiring and comparing whole-brain connectivity signatures, as outlined above, in healthy subjects and subjects who might be at risk of developing a neuropsychiatric disease or disorder, for example due to genetic predisposition, or who might indicate (early) phenotypical signs of dementia, a neuropsychiatric disease or disorder might be detected and diagnosed before phenotypical signs appear. In case of a neurodegenerative disease or disorder, it might be detected and diagnosed already after early phenotypical signs of dementia have been observed and might so aid the medical practitioner in selecting and deciding on the most suitable timing and course of treatment. Furthermore, the effectiveness of a neuropsychiatric treatment regimen in subjects suffering from a neuropsychiatric disease or disorder can be evaluated and monitored using whole-brain functional connectivity analysis, for example, by analysis before and after treatment or by analysis on treatment versus off treatment.

Moreover, a quick assessment of whole-brain connectivity signatures might serve as a rapidly available clinical diagnostic marker in the emergency room setting to discern subjects with a neuropsychiatric disease or disorder from subjects who just suffered a stroke.

We anticipate that this approach will prove useful both in diagnosing subjects suffering from specific disorders and also as an objective measure of treatment response in clinical trials where changes in whole-brain functional connectivity patterns would be expected to reflect and possibly precede behavioral or cognitive improvements. Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Beckmann C F et al. (2005) Investigations into resting-state connectivity using independent component analysis. Philos Trans R Soc Lond B Biol Sci 360(1457):1001-1013.

Cohen A L et al. (2008). Defining functional areas in individual human brains using resting functional connectivity MRI. Neuroimage 41:45-57.

Damoiseaux J S et al. (2006) Consistent resting-state networks across healthy subjects. Proc Natl Acad Sci USA 103(37):13848-13853.

Davatzikos C et al. (2005). Classifying spatial patterns of brain activity with machine learning methods: application to lie detection. Neuroimage 28:663-668.

Dubois et al. (2007). “Research criteria for the diagnosis of Alzheimer's disease: revising the NINCDS-ADRDA criteria”. Lancet Neurol 6:734-746.

Fox M D et al. (2005). The human brain is intrinsically organized into dynamic, anticorrelated functional networks. Proc Natl Acad Sci USA 102:9673-9678.

Friston K J et al. (1995). Spatial registration and normalization of images. Human Brain Mapping 3:165-189.

Glover G H & Lai S (1998). Self-navigated spiral fMRI: interleaved versus single-shot. Magn Reson Med 39:361-368.

Glover G H & Law C S (2001). Spiral-in/out BOLD fMRI for increased SNR and reduced susceptibility artifacts. Magn Reson Med 46:515-522.

Greicius M D et al. (2003) Functional connectivity in the resting brain: a network analysis of the default mode hypothesis. Proc Natl Acad Sci USA 100(1):253-258.

Greicius M D et al. (2004) Default-mode network activity distinguishes Alzheimer's disease from healthy aging: evidence from functional MRI. Proc Natl Acad Sci USA 101(13):4637-4642.

Greicius M D et al. (2009). Resting-state functional connectivity reflects structural connectivity in the default mode network. Cereb Cortex 19:72-78.

Hastie T et al. (2009). The Elements of Statistical Learning 2nd edn New York, N.Y.: Springer.

James W (1918). The Principles of Psychology. Vol 1 New York, N.Y.: Holt and Company. Kim D H et al. (2002). Regularized higher-order in vivo shimming. Magn Reson Med 48:715-722.

Kiviniemi V et al. (2009). Functional segmentation of the brain cortex using high model order group PICA. Hum Brain Mapp 30:3865-3886.

Li K et al. (2009). Review of methods for functional brain connectivity detection using fMRI. Computerized Medical Imaging and Graphics 33:131-139.

Mitchell T M et al. (2003). Classifying instantaneous cognitive states from FMRI data. AMIA Annu Symp Proc 465-469.

Mourao-Miranda J et al. (2005). Classifying brain states and determining the discriminating activation patterns: Support Vector Machine on functional MRI data. Neuroimage 28: 980-995.

Owen A M et al. (2006). Detecting awareness in the vegetative state. Science 313:1402.

Poline J B et al. (1997). Combining spatial extent and peak intensity to test for activations in functional imaging. Neuroimage 5:83-96.

Rushworth M F et al. (2006). Connection patterns distinguish 3 regions of human parietal cortex. Cereb Cortex 16:1418-1430.

Seeley W W et al. (2007). Dissociable intrinsic connectivity networks for salience processing and executive control. J Neurosci 27(9):2349-2356.

Smith S M et al. (2009). Correspondence of the brain's functional architecture during activation and rest. Proc Natl Acad Sci USA 106:13040-13045.

Supekar K et al. (2008). Network analysis of intrinsic functional brain connectivity in Alzheimer's disease. PLoS Comput Biol 4(6):e1000100.

Tzourio-Mazoyer N et al. (2002). Automated anatomical labeling of activations in SPM using a macroscopic anatomical parcellation of the MNI MRI single-subject brain. Neuroimage 15:273-289.

Zhou J et al. (2010). Divergent network connectivity changes in behavioural variant frontotemporal dementia and Alzheimer's disease. Brain 133(Pt 5):1352-1367.

What is claimed is:

1. A method of classifying specific cognitive states in a subject, the method comprising
(a) obtaining whole-brain functional images from a subject during resting state (resting state matrix), before or after whole brain functional images were obtained from said subject during at least one state of task;
(b) obtaining whole-brain functional images from a subject during at least one state of task (task state matrix);
(c) defining regions of interest (ROIs) from said whole-brain functional images during resting state;
(d) creating a difference matrix by overlaying and subtracting said resting state matrix from said task state matrix;
(e) obtaining whole-brain connectivity markers through correlating said regions of interest;
(f) analyzing said whole-brain connectivity markers;
(g) physical transformation of said whole-brain connectivity markers into information for graphical display or output to a computer-readable medium, computer or computer network.

2. A method of diagnosing a neuropsychiatric disease in a subject using whole-brain connectivity signatures, the method comprising
(a) obtaining whole-brain functional images from a subject during resting state (resting state matrix),
(b) assessing connectivity between a set of regions of interest (ROIs) from said whole-brain functional images during resting state to obtain whole-brain connectivity markers from said subject;
(c) analyzing said whole-brain connectivity markers in said subject in comparison to whole-brain connectivity markers obtained from a group of healthy control subjects and a group of subjects suffering from a given neuropsychiatic disease for variations as a basis for diagnosing a neuropsychiatric disease;
(d) physical transformation of said whole-brain connectivity markers into information for graphical display or output to a computer-readable medium, computer or computer network.

3. The method of claim 2, wherein the neuropsychiatric disease is a neurodegenerative disease such as Alzheimer's disease, Parkinson's disease, Lewy body dementia, Huntington's disease, a tauopathy, a serpinopathy, a prion disease, frontotemporal or vascular dementia.

4. The method of claim 2, wherein the neuropsychiatric disease is chronic pain, depression or anxiety.

5. A method of monitoring progression of a neuropsychiatric disease in a subject using whole-brain connectivity signatures, the method comprising, over a predetermined period of time and repeatedly,
(a) obtaining whole-brain functional images from a subject during resting state (resting state matrix).
(b) assessing connectivity between a set of regions of interest (ROIs) from said whole-brain functional images during resting state to obtain whole-brain connectivity markers from said subject;
(c) analyzing said whole-brain connectivity markers to monitor progression of a neuropsychiatric disease in said subject in comparison to whole-brain connectivity markers obtained from said subject at one or more earlier timepoints and optionally in comparison to whole-brain connectivity markers obtained from a group of healthy control subjects;
(d) physical transformation of said whole-brain connectivity markers into information for graphical display or output to a computer-readable medium, computer or computer network.

6. The method of claim 5, wherein the neuropsychiatric disease is a neurodegenerative disease such as Alzheimer's disease, Parkinson's disease, Lewy body dementia, Huntington's disease, a tauopathy, a serpinopathy, a prion disease, frontotemporal or vascular dementia.

7. The method of claim 5, wherein the neuropsychiatric disease is chronic pain, depression or anxiety.

8. A method of monitoring treatment success of a neuropsychiatric disease in a subject using whole-brain connectivity signatures, the method comprising, over a predetermined period of time and repeatedly,
(a) obtaining whole-brain functional images from a subject during resting state (resting state matrix);
(b) assessing connectivity between a set of regions of interest (ROIs) from said whole-brain functional images during resting state to obtain whole-brain connectivity markers from said subject;
(c) analyzing said whole-brain connectivity markers to monitor treatment success of a neuropsychiatric disease in said subject in comparison to whole-brain connectivity markers obtained from said subject at one or more later timepoints following a treatment intervention and optionally in comparison to whole-brain connectivity markers obtained from a group of healthy control subjects;
(e) physical transformation of said whole-brain connectivity markers into information for graphical display or output to a computer-readable medium, computer or computer network.

9. The method of claim 8, wherein the neuropsychiatric disease is a neurodegenerative disease such as Alzheimer's disease, Parkinson's disease, Lewy body dementia, Huntington's disease, a tauopathy, a serpinopathy, a prion disease, frontotemporal or vascular dementia.

10. The method of claim 8, wherein the neuropsychiatric disease is chronic pain, depression or anxiety.

11. A method of predicting consumer behavior by classifying specific cognitive states in a subject, the method comprising
(a) obtaining whole-brain functional images from a subject during resting state (resting state matrix), before or after whole-brain functional images were obtained from said subject during exposure to images of a commercial product;
(b) obtaining whole-brain functional images from a subject during exposure to images of a commercial product (product matrix);
(c) defining regions of interest (ROIs) from said whole-brain functional images during resting state;
(d) creating a difference matrix by overlaying and subtracting said resting state matrix from said product matrix;
(e) obtaining whole-brain connectivity markers through correlating said regions of interest;
(f) analyzing said whole-brain connectivity markers;
(g) physical transformation of said whole-brain connectivity markers into information for graphical display or output to a computer-readable medium, computer or computer network.

12. A method of predicting financial decision making by classifying specific cognitive states in a subject, the method comprising
(a) obtaining whole-brain functional images from a subject during resting state (resting state matrix), before or after whole-brain functional images were obtained from said subject during at least one state of financial decision making task (task state matrix;
(b) obtaining whole-brain functional images from a subject during at least one state of financial decision making task (task state matrix;
(c) defining regions of interest (ROIs) from said whole-brain functional images during resting state;
(d) creating a difference matrix by overlaying and subtracting said resting state matrix from said task state matrix;
(e) obtaining whole-brain connectivity markers through correlating said regions of interest;
(f) analyzing said whole-brain connectivity markers;
(g) physical transformation of said whole-brain connectivity markers into information for graphical display or output to a computer-readable medium, computer or computer network.
